(19) United States
(12) Patent Application Publication
(10) Pub. No.: US 2017/0235902 A1
(43) Pub. Date: Aug. 17, 2017

(54) METHOD AND SYSTEM FOR CHARACTERIZATION OF CLOSTRIDIUM DIFFICILE ASSOCIATED CONDITIONS

(71) Applicant: uBiome, Inc., San Francisco, CA (US)

(72) Inventors: Daniel Almonacid, San Francisco, CA (US); Zachary Apte, San Francisco, CA (US); Jessica Richman, San Francisco, CA (US)

(21) Appl. No.: 15/374,890

(22) Filed: Dec. 9, 2016

Related U.S. Application Data
(63) Continuation-in-part of application No. 15/097,862, filed on Apr. 13, 2016.
(60) Provisional application No. 62/146,810, filed on Apr. 13, 2015.
(60) Provisional application No. 62/146,833, filed on Apr. 13, 2015.
(60) Provisional application No. 62/147,124, filed on Apr. 14, 2015.
(60) Provisional application No. 62/146,852, filed on Apr. 13, 2015.
(60) Provisional application No. 62/147,058, filed on Apr. 14, 2015.
(60) Provisional application No. 62/147,077, filed on Apr. 14, 2015.
(60) Provisional application No. 62/147,315, filed on Apr. 14, 2015.
(60) Provisional application No. 62/147,337, filed on Apr. 14, 2015.
(60) Provisional application No. 62/265,077, filed on Dec. 9, 2015.

(51) Int. Cl. G06F 19/00; G06F 19/12; C12Q 1/68; G06F 19/22

(52) U.S. Cl. CPC G06F 19/345; G06F 19/22; G06F 19/12; G06F 19/3437; C12Q 1/689; C12Q 1/6869

(57) ABSTRACT

An embodiment of a system and method for characterizing a Clostridium-associated condition in relation to a user includes: a handling network operable to receive containers including material from a set of users, the handling network including a sequencing system operable to determine microbiome sequences from sequencing the material; a processing system operable to generate a microbiome composition dataset and a microbiome functional diversity dataset based on the microbiome sequences, receive a supplementary dataset associated with the Clostridium-associated condition for the set of users; transform the supplementary dataset and features extracted from the microbiome composition dataset and the microbiome functional diversity dataset into a characterization model for the Clostridium-associated condition; and a therapy system operable to promote a therapy to the user based on characterizing the user in relation to the Clostridium-associated condition using the characterization model.

### CROSS-REFERENCE TO RELATED APPLICATIONS

This application is a continuation-in-part of U.S. patent application Ser. No. 15/097,862, filed on 13 Apr. 2016, which claims the benefit of U.S. Provisional Application Ser. No. 62/146,810 filed 13 Apr. 2015, U.S. Provisional Application Ser. No. 62/146,833 filed 13 Apr. 2015, U.S. Provisional Application Ser. No. 62/147,124 filed 14 Apr. 2015, U.S. Provisional Application Ser. No. 62/146,852 filed 13 Apr. 2015, U.S. Provisional Application Ser. No. 62/147,058 filed 14 Apr. 2015, U.S. Provisional Application Ser. No. 62/147,077 filed 14 Apr. 2015, U.S. Provisional Application Ser. No. 62/147,315 filed 14 Apr. 2015, and U.S. Provisional Application Ser. No. 62/147,337 filed 14 Apr. 2015, which are each incorporated in their entirety herein by this reference.

This application also claims the benefit of U.S. Provisional Application Ser. No. 62/265,077 filed 9 Dec. 2015, which is incorporated in its entirety herein by this reference.

### TECHNICAL FIELD

This invention relates generally to the field of microbiology and more specifically to a new and useful system and method for characterizing Clostridium difficile-associated conditions in the field of microbiology.

### BACKGROUND

A microbiome is an ecological community of commensal, symbiotic, and pathogenic microorganisms that are associated with an organism. The human microbiome includes over 10 times more microbial cells than human cells, but characterization of the human microbiome is still in nascent stages due to limitations in sample processing techniques, genetic analysis techniques, and resources for processing large amounts of data. Nonetheless, the microbiome is suspected to play at least a partial role in a number of health/disease-related states (e.g., preparation for childbirth, diabetes, auto-immune disorders, gastrointestinal disorders, rheumatoid disorders, neurological disorders, etc.). Given the profound implications of the microbiome in affecting a subject's health, efforts related to the characterization of the microbiome, the generation of insights from the characterization, and the generation of therapeutics configured to rectify states of dysbiosis should be pursued. Current methods and systems for analyzing the microbiomes of humans and providing therapeutic measures based on gained insights have, however, left many questions unanswered. In particular, methods for characterizing certain health conditions and therapies (e.g., probiotic therapies) tailored to specific subjects based upon microbiome composition and/or functional features have not been viable due to limitations in current technologies.

As such, there is a need in the field of microbiology for a new and useful system and method for characterizing Clostridium difficile (C. difficile) associated conditions in an individualized and population-wide manner. This invention creates such a new and useful system and method.

### BRIEF DESCRIPTION OF THE FIGURES

FIGS. 1A-1B are flowcharts of an embodiment of a method for microbiome characterization;

FIG. 2 depicts an embodiment of a system and method for microbiome characterization;

FIG. 3 depicts a variation of a process for generation of a model in an embodiment of a system and method for microbiome characterization;

FIG. 4 depicts variations of mechanisms by which probiotic-based therapies operate in an embodiment of a method for microbiome characterization;

FIG. 5 depicts examples of notification provision in an example of a method for microbiome characterization;

FIG. 6 depicts a variation of an interface for providing Clostridium-associated condition-related information in an example of a method for microbiome characterization;

FIG. 7 depicts examples of notification provision in an example of a method for microbiome characterization; and

FIG. 8 depicts examples of notification provision in an example of a method for microbiome characterization.

### DESCRIPTION OF THE EMBODIMENTS

The following description of the embodiments of the invention is not intended to limit the invention to these embodiments, but rather to enable any person skilled in the art to make and use this invention.

### 1. Overview.

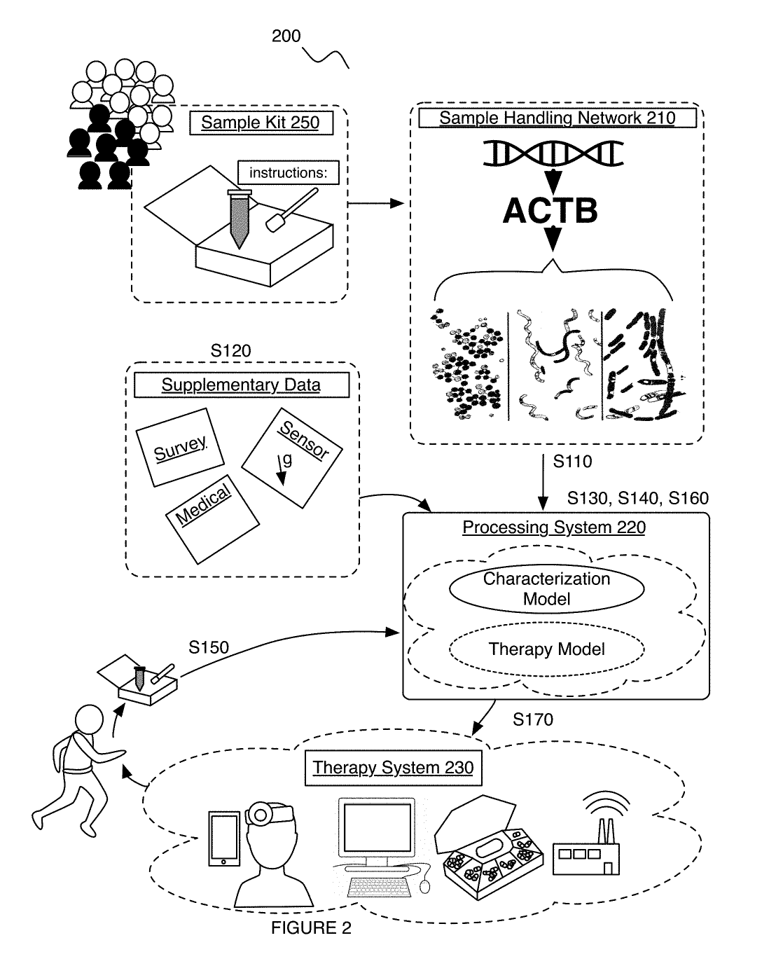

As shown in FIG. 2, an embodiment of a system 200 for characterizing a Clostridium-associated condition in relation to a user (e.g., a human subject, an animal subject, etc.) includes: a handling network (e.g., sample handling network) 210 operable to receive containers including material (e.g., biological samples including microorganism nucleic acid material, etc.) from a set of users (e.g., a population of users), the handling network including a sequencing system operable to determine microbiome sequences from sequencing the material; a processing system 220 operable to generate a microbiome composition dataset and a microbiome functional diversity dataset based on the microbiome sequences, receive a supplementary dataset associated with the Clostridium-associated condition for the set of users; transform the supplementary dataset and features extracted from the microbiome composition dataset and the microbiome functional diversity dataset into a characterization model for the Clostridium-associated condition; and a therapy system 230 operable to promote a therapy to the user based on characterizing the user in relation to the Clostridium-associated condition using the characterization model.

The system 200 can additionally or alternatively include one or more of: an interface 240 operable to present Clostridium-associated condition-related information; a sample kit 250 functioning to provide a subject with the components and/or instructions to collect and/or process a biological sample from one or more collection sites of the subject; and/or any other suitable component.

The system 200 and/or method 100 function to generate and/or apply models (e.g., characterization models, therapy models, etc.) that can be used to characterize (e.g., diagnose) subjects according to at least one of their microbiome composition and functional features (e.g., as a clinical diagnostic, as a companion diagnostic, etc.), and/or provide therapeutic measures (e.g., probiotic-based therapeutic measures, phage-based therapeutic measures, small-molecule-based therapeutic measures, fecal matter transplant-based therapeutic measures, clinical measures, etc.) to subjects based upon microbiome characterization for a population of subjects.

As such, data from the population of subjects can be used to characterize subjects according to their microbiome composition and/or functional features, indicate states of health and areas of improvement based upon the characterization(s), and promote one or more therapies that can modulate the composition of a subject's microbiome toward one or more of a set of desired equilibrium states. Variations of the method 100 can further facilitate monitoring and/or adjusting of therapies provided to a subject, for instance, through reception, processing, and analysis of additional samples from a subject throughout the course of therapy. In specific examples, the system 200 and/or the method 100 can be used to promote targeted therapies to subjects suffering from various health conditions.

The system 200 and/or components of the system 200 preferably implement the method 100 and/or portions of the method 100, but any suitable components can partially and/or fully implement the method 100. The method 100 can be implemented for a single subject for whom microbiome characterization and/or microbiome modulation with therapeutics is of interest, and can additionally or alternatively be implemented for a population of subjects (e.g., including the subject, excluding the subject), where the population of subjects can include patients dissimilar to and/or similar to the subject (e.g., in health condition, in dietary needs, in demographic features, etc.). Thus, information derived from the population of subjects can be used to provide additional insight into connections between behaviors of a subject and effects on the subject's microbiome, due to aggregation of data from a population of subjects.

In implementation of the method 100, an aggregate set of biological samples is preferably received from a wide variety of subjects, collectively including subjects of one or more of: different demographics (e.g., genders, ages, marital statuses, ethnicities, nationalities, socioeconomic statuses, sexual orientations, etc.), different health conditions (e.g., health and disease states), different living situations (e.g., living alone, living with pets, living with a significant other, living with children, etc.), different dietary habits (e.g., omnivorous, vegetarian, vegan, sugar consumption, acid consumption, etc.), different behavioral tendencies (e.g., levels of physical activity, drug use, alcohol use, etc.), different levels of mobility (e.g., related to distance traveled within a given time period), and/or any other suitable trait that has an effect on microbiome composition and/or functional features. As such, as the number of subjects increases, the predictive power of processes implemented in blocks of the method 100 increases, in relation to characterizing a variety of subjects based upon their microbiomes. However, the method 100 can involve generation of diagnostic and therapies derived from biological sample data from any other suitable group of subjects.

In a variation, the system 200 can be used to implement an embodiment of a method 100 for characterizing a Clostridium difficile (C. difficile) associated condition in relation to a user including: generating a microbiome composition dataset and a microbiome functional diversity dataset based on nucleic acid sequences derived from material (e.g., biological sample including nucleic acid material) from a set of users (e.g., population of users); receiving a supplementary dataset informative of the C. difficile associated condition for the set of users; obtaining a set of C. difficile associated feature-selection rules correlating the C. difficile associated condition to a subset of microbiome composition features and a subset of microbiome functional diversity features; generating a feature set based on evaluating the microbiome composition dataset and the microbiome functional diversity dataset against the set of C. difficile associated feature-selection rules; applying the feature set with the supplementary dataset to generate a characterization model for the C. difficile associated condition; generating a characterization of the user in relation to the C. difficile associated condition using the characterization model; and promoting a therapy to the user based on the characterization.

### 2. Benefits.

The onset of sequencing technologies (e.g., next-generation sequencing) has given rise to technological issues (e.g., data processing issues, information display issues, microbiome analysis issues, therapy prediction issues, etc.) that would not exist but for the unprecedented advances in speed and data generation associated with sequencing nucleic acid material. Examples of the system 200 and the method 100 confer technologically-rooted solutions to at least such technological issues.

First, the technology can confer improvements in computer-related technology (e.g., artificial intelligence, machine learning, etc.) by facilitating computer performance of functions not previously performable. For example, the technology can computationally generate microbiome characterizations and recommended therapies associated with Clostridium-associated conditions, based on microbiome sequence datasets and microorganism reference sequence databases (e.g., Genome Reference Consortium) that are recently viable due to advances in sample processing techniques and sequencing technology. The microbial cells constituting a human microbiome can be over ten times larger than human cells, which can translate into a plethora of data giving rise to issues of processing and analysis to generate actionable microbiome insights in relation to potentially life-threatening Clostridium-associated conditions (e.g., sepsis, colitis, etc.).

Second, the technology can confer improvements in processing speed and microbiome characterization accuracy. The technology can generate and apply Clostridium-associated condition feature-selection rules to select an optimized subset of features (e.g., microbiome composition features, microbiome functional diversity features, etc.) out of a vast potential pool of features (e.g., extractable from the plethora of microbiome data) for generating and applying characterization models and/or therapy models. The Clostridium-associated condition feature-selection rules can thus enable shorter training and execution times (e.g., for predictive machine learning models), model simplification facilitating efficient interpretation of results, reduction in overfitting, and other suitable improvements.

Third, the technology can transform entities (e.g., users, biological samples, therapy systems including medical devices, etc.) into different states or things. For example, the system 200 and/or method 100 can identify therapies to promote to a patient to modify microbiome composition and/or function to prevent and/or ameliorate Clostridium-associated conditions, thereby transforming the microbiome of the patient. In another example, the technology can transform biological samples received by a population of patients into microbiome datasets usable in generating characterization models and/or therapy models. In another example, the technology can control therapy systems to promote therapies (e.g., by generating control instructions for the therapy system to execute), thereby transforming the therapy system. The technology can, however, provide any other suitable benefit(s) in the context of using non-generalized computer systems for characterizing a microbiome and/or promoting a relevant therapy.

### 3. System.

The sample handling network 210 of the system 200 functions to receive and process (e.g., fragment, amplify, sequence, etc.) biological samples to transform microorganism nucleic acids of the biological samples into genetic sequences that can be subsequently aligned and analyzed to generate characterizations of and therapies for Clostridium-associated conditions. The sample handling network 210 can additionally or alternatively function to provide a sample kit 250 (e.g., including sample containers, instructions, etc.) to a user (e.g., in response to a purchase order for a sample kit 250), such as through a mail delivery system.

The sample handling network 210 can additionally or alternatively include a library preparation system operable to automatically prepare biological samples (e.g., fragment and amplify using primers compatible with microbiome targets associated with the Clostridium-associated condition) in a multiplex manner to be sequenced by a sequencing system; and/or a sequencing system (e.g., MiSeq/NextSeq/HiSeq and/or other suitable sequencing platform) operable to sequence nucleic acids (e.g., microorganism DNA and/or RNA) derived from biological samples received at the sample handling network 210. The sample handling network 210 is preferably remote from a user, such that a user can conveniently send a collected biological sample to the sample handling network 210, and digitally receive results based on the collected biological sample. Additionally or alternatively, the sample handling network 210 can include user action (e.g., a user pre-processing a sample), a user device (e.g., an application executing on a mobile device that aids in analysis of the sample), a remote server, and/or any other suitable entity. However, the sample handling network 210 can be configured in any suitable manner.

The processing system 220 of the system 200 functions to analyze a dataset (e.g., a microbiome sequence dataset) derived from a processed sample to generate and/or apply a characterization model for characterizing one or more Clostridium-associated conditions. The processing system 220 can additionally or alternatively function to generate and/or apply a therapy model for identifying a therapy used to treat a Clostridium-associated condition; to promote the therapy (e.g., acting as a therapy system 230 to generate and/or output a therapy recommendation to a subject at a user device); and/or perform any suitable function (e.g., any portion of the method 100). For example, the processing system 220 can be operable to obtain a set of Clostridium feature-selection rules correlating the condition to subsets of composition features and functional diversity features; and generate a feature set (e.g., used in generating a characterization model) based on applying the rules to one or more microbiome datasets. Such feature-selection rules can improve the processing system 220 by facilitating decreased processing time to transform the features and/or other suitable data (e.g., a supplementary dataset) into the characterization model (e.g., by training the model using training data labels derived from the supplementary dataset).

The processing system 220 and/or other components of the system 200 can additionally or alternatively include and/or communicate data to and/or from: a remote computing system (e.g., remote servers, cloud systems, etc.), a local computing system, a user database (e.g., storing user account information, characterization information such as for a Clostridium-associated condition, user health records, user demographic information, associated care provider information, associated guardian information, user device information, etc.), an analysis database (storing models, collected data, historical data, public data, simulated data, generated datasets, generated analyses, diagnostic results, therapy recommendations, etc.), user device (e.g., a smartphone executing an application for storing and/or executing a characterization and/or therapy model, etc.), a care provider device (e.g., a device of a care provider associated with a user), a machine configured to receive a computer-readable medium storing computer-readable instructions, and/or any other suitable component. However, the processing system 220 can be configured in any suitable manner.

The therapy system 230 of the system 200 functions to promote one or more therapies (e.g., identified by the a therapy model generated and/or executed by the processing system 220) to a subject or a care provider to implement in ameliorating and/or preventing a Clostridium-associated condition. The therapy system 230 can additionally or alternatively function to monitor efficacy of one or more therapies to, for example, generate data that can be used in updating a model (e.g., a therapy model). The therapy system 230 can include any one or more of: a communications system (e.g., to communicate therapy recommendations to a user device and/or care provider device; to enable telemedicine between a care provider and a subject in relation to a Clostridium-associated condition; etc.), an application executable on a user device (e.g., a dietary regimen application for recommending microbiome composition modification therapies, etc.), a medical device (e.g., a central venous catheter for administering medication and/or fluids; colonoscopy devices, sigmoidoscopy devices, and/or other screening devices; a biometric sensor for monitoring biometric data related to a Clostridium-associated condition, such as C. difficile toxin A or B; a biological sampling device, such as for sampling stool of a subject; etc.), a user device (e.g., biometric sensors of a user smartphone operable to collect biometric data associated with a Clostridium-associated condition), and/or any other suitable component. The therapy system 230 is preferably controllable by the processing system 220. For example, the processing system 220 can generate control instructions to transmit to the therapy system 230 to execute to promote the therapy. In another example, the processing system 220 can update and/or otherwise modify an application and/or other therapy system software of a device (e.g., user smartphone) to promote a therapy (e.g., promoting, at a to-do application, lifestyle changes for modifying microbiome functional diversity to reduce the risk of C. difficile-based colitis infection). However, the therapy system 230 can be configured in any other manner.

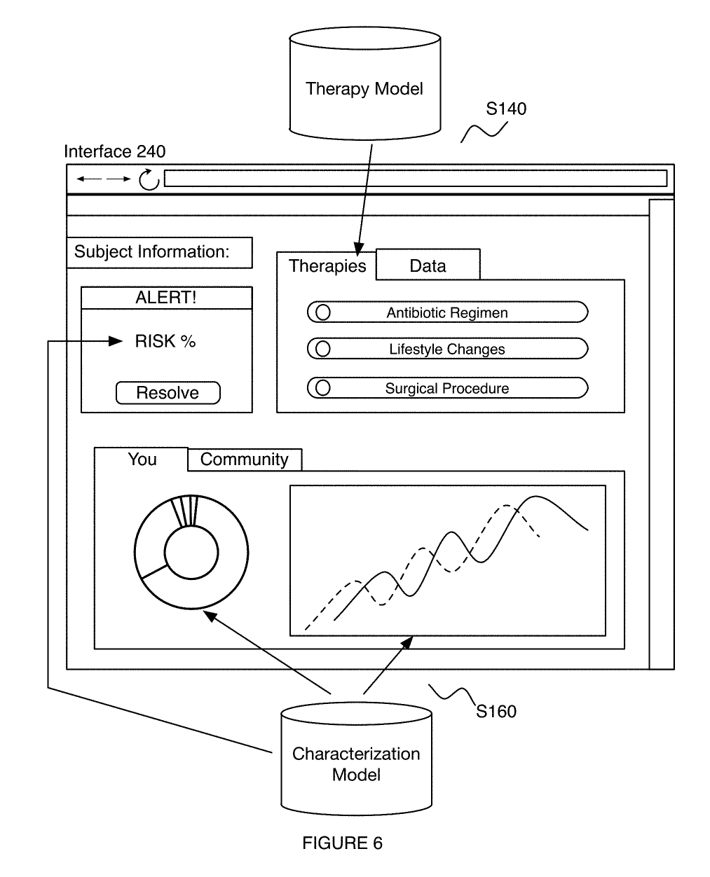

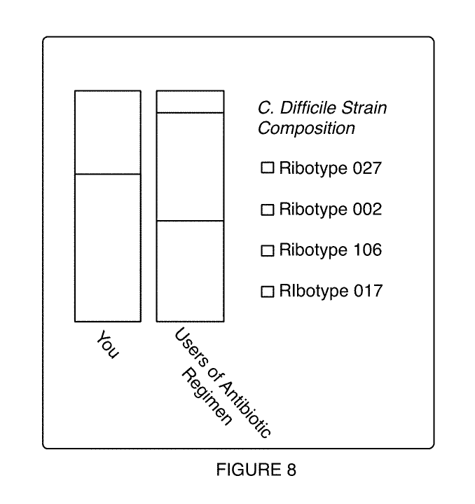

As shown in FIG. 6, the system 200 can additionally or alternatively include an interface 240 functioning to improve the presentation of Clostridium-associated condition-related information (e.g., characterizations, therapy recommendations, etc.) at a user device and/or care provider device (e.g., remotely accessing the interface 240 through an application, at a website, at a document, etc.). The interface 240 can be a user interface (e.g., for presentation to a subject), a care provider interface, and/or any other suitable interface 240. The interface 240 preferably includes a plurality of displays (e.g., a first display introducing microbiome composition and/or microbiome functional diversity information; a second display analyzing the information, etc.), but can include any number of displays configured in any manner. The interface 240 can present information in a verbal, numerical, graphical, audio, and/or any suitable format of information. The presented information can include and/or be associated with one or more of: microbiome composition, microbiome functional diversity, Clostridium-associated condition-related information (e.g., presence and/or risk of Clostridium microorganisms and/or infection, etc.), behavioral characteristics, demographic characteristics, individual characteristics, comparisons with other subjects and/or demographics (e.g., comparing risk of Clostridium infection between the user and a group of smokers, etc.), population characteristics, and/or any other suitable information. In an example, the interface 240 can present a microbiome composition for the user relative to a user group sharing a demographic characteristic, where the microbiome composition includes taxonomic groups including at least one of C. difficile, Clostridium botulinum, and Clostridium perfringens. In another example, as shown in FIG. 8, the interface 240 can present a microbiome composition detailing the relative abundance of different Clostridium strains (e.g., different C. difficile strains), which can possess varying correlations with Clostridium infections (e.g., a higher incidence of Clostridium-based sepsis from C. difficile Ribotype 027 strain compared to other C. difficile strains).

In a variation, the interface 240 can automatically highlight portions of the presented information, such as through one or more of: resizing operations (e.g., graphics, text, etc. for fitting information within the screen dimensions of a particular device, and/or other suitable purpose), color modification (e.g., using yellow highlighting for therapy recommendations, etc.), disability accommodation (e.g., translating text into audio), and/or other suitable operations. Highlighting presented information can function to guide a subject and/or care provider through an analysis of the presented information. In another variation, the interface 240 can facilitate user interaction with the interface 240. For example, the interface 240 can provide options for selecting different demographic groups (e.g., hospital patients, recently released hospital patients, exercisers, smokers, consumers of probiotics, antibiotic users, groups undergoing particular therapies, etc.) to compare (e.g., through charts and graphs) microbiome composition, functional diversity, and/or other Clostridium-associated condition-related information. In other examples, the interface 240 can provide a log (e.g., for logging lifestyle habits, therapy regimens, etc.), digital surveys (e.g., for inquiring about symptoms associated with Clostridium infections), therapies, and/or other suitable components that can, for example, be used in updating characterizations, therapies, models, and/or other suitable data. However, the interface 240 can be configured in any suitable manner.

In variations, any of the components of the system 200 can perform functions associated with other components. For example, the processing system 220 can perform sequencing functions associated with the sequencing system; generate characterizations of and therapies for a Clostridium-associated condition; and promote the therapies (e.g., through generating and transmitting therapy-related notifications to a subject). Additionally or alternatively, the system 200 and/or method 100 can include any suitable components and/or functions analogous to those described in U.S. application Ser. No. 14/593,424 filed 9 Jan. 2015, U.S. application Ser. No. 15/198,818 filed 30 Jun. 2016, U.S. application Ser. No. 15/098,027 filed 13 Apr. 2016, U.S. application Ser. No. 15/098,248 filed 13 Apr. 2016, U.S. application Ser. No. 15/098,236 filed 13 Apr. 2016, Ser. No. 15/098,222 filed 13 Apr. 2016, U.S. application Ser. No. 15/098,204 filed 13 Apr. 2016, U.S. application Ser. No. 15/098,174 filed 13 Apr. 2016, U.S. application Ser. No. 15/098,110 filed 13 Apr. 2016, U.S. application Ser. No. 15/098,081 filed 13 Apr. 2016, U.S. application Ser. No. 15/098,153 filed 13 Apr. 2016, U.S. application Ser. No. 15/228,890 filed 4 Aug. 2016, and U.S. application Ser. No. 15/240,919 filed 18 Aug. 2016, which are each hereby incorporated in their entirety by this reference. However, the components of the system 200 can be configured in any suitable manner.

### 4. Method.

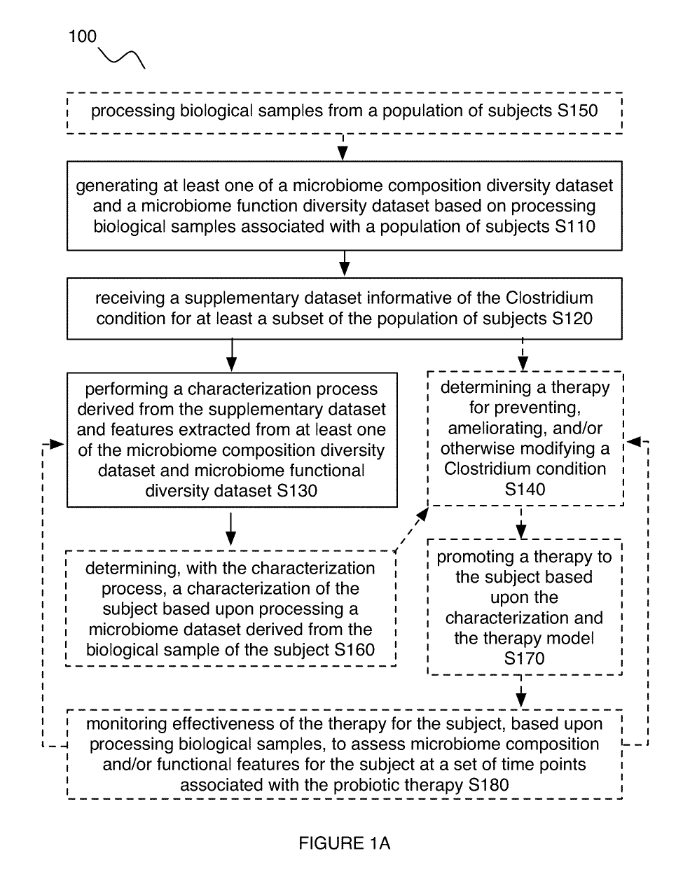

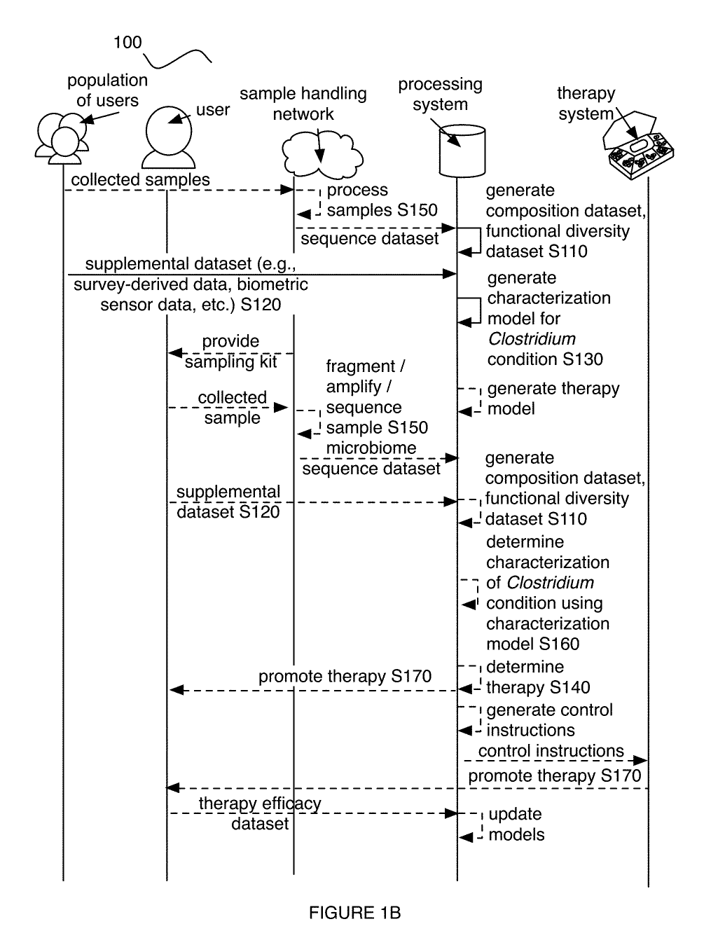

As shown in FIGS. 1A-1B, a method 100 for characterizing a Clostridium-associated condition (e.g., a C. difficile condition) in relation to a subject includes: generating at least one of a microbiome composition dataset and a microbiome function diversity dataset based on processing biological samples associated with a population of subjects S110; receiving a supplementary dataset informative of the Clostridium-associated condition for at least a subset of the population of subjects S120; and performing a characterization process derived from the supplementary dataset and features extracted from at least one of the microbiome composition dataset and microbiome functional diversity dataset S130. The method 100 can additionally or alternatively include: determining a therapy for preventing, ameliorating, and/or otherwise modifying a Clostridium-associated condition S140; processing a biological sample from a subject S150; determining, with the characterization process, a characterization of the subject based upon processing a microbiome dataset (e.g., microbiome composition dataset, microbiome functional diversity dataset, etc.) derived from the biological sample of the subject S160; promoting a therapy to the subject based upon the characterization and the therapy model S170; monitoring effectiveness of the therapy for the subject, based upon processing biological samples, to assess microbiome composition and/or functional features for the subject at a set of time points associated with the probiotic therapy S180; and/or any other suitable operations.

Block S110 recites: generating at least one of a microbiome composition dataset and a microbiome function diversity dataset based on processing biological samples associated with a population of subjects. Block S110 functions to process each of an aggregate set of biological samples, in order to determine compositional and/or functional aspects associated with the microbiome of each of a population of subjects. Compositional and functional aspects can include compositional aspects at the microorganism level, including parameters related to distribution of microorganisms across different groups of kingdoms, phyla, classes, orders, families, genera, species, subspecies, strains, and/or any other suitable infraspecies taxon (e.g., as measured in total abundance of each group, relative abundance of each group, total number of groups represented, etc.). Compositional and functional aspects can also be represented in terms of operational taxonomic units (OTUs). Compositional and functional aspects can additionally or alternatively include compositional aspects at the genetic level (e.g., regions determined by multilocus sequence typing, 16S rRNA sequences, 18S rRNA sequences, ITS sequences, other genetic markers, other phylogenetic markers, etc.). Compositional and functional aspects can include the presence or absence or the quantity of genes associated with specific functions (e.g. enzyme activities, transport functions, immune activities, etc.). Outputs of Block S110 can thus be used to provide features of interest for the characterization process of Block S130 and/or the therapy process of Block S140, where the features can be microorganism-based (e.g., presence of a genus of bacteria), genetic-based (e.g., based upon representation of specific genetic regions and/or sequences) functional-based (e.g., presence of a specific catalytic activity), and/or otherwise configured.

In one variation, Block S110 can include assessment and/or processing based upon phylogenetic markers derived from bacteria and/or archaea in relation to gene families associated with one or more of: ribosomal protein S2, ribosomal protein S3, ribosomal protein S5, ribosomal protein S7, ribosomal protein S8, ribosomal protein S9, ribosomal protein S10, ribosomal protein S11, ribosomal protein S12/S23, ribosomal protein S13, ribosomal protein S15P/S13e, ribosomal protein S17, ribosomal protein S19, ribosomal protein L1, ribosomal protein L2, ribosomal protein L3, ribosomal protein L4/L1e, ribosomal protein L5, ribosomal protein L6, ribosomal protein L10, ribosomal protein L11, ribosomal protein L13, ribosomal protein L14b/L23e, ribosomal protein L15, ribosomal protein L16/L10E, ribosomal protein L18P/L5E, ribosomal protein L22, ribosomal protein L24, ribosomal protein L25/L23, ribosomal protein L29, translation elongation factor EF-2, translation initiation factor IF-2, metalloendopeptidase, ffh signal recognition particle protein, phenylalanyl-tRNA synthetase beta subunit, phenylalanyl-tRNA synthetase alpha subunit, tRNA pseudouridine synthase B, porphobilinogen deaminase, phosphoribosylformylglycinamidine cyclo-ligase, and ribonuclease HII. However, the markers can include any other suitable marker(s).

For Block S110, characterizing the microbiome composition and/or functional features for each of the aggregate set of biological samples thus preferably includes a combination of sample processing techniques (e.g., wet laboratory techniques) and computational techniques (e.g., utilizing tools of bioinformatics) to quantitatively and/or qualitatively characterize the microbiome and functional features associated with each biological sample from a subject or population of subjects. In variations, sample processing in Block S110 can include any one or more of: lysing a biological sample, disrupting membranes in cells of a biological sample, separation of undesired elements (e.g., RNA, proteins) from the biological sample, purification of nucleic acids (e.g., DNA) in a biological sample, amplification (e.g., with a library preparation system) of nucleic acids from the biological sample, further purification of amplified nucleic acids of the biological sample, sequencing (e.g., with a sequencing system) of amplified nucleic acids of the biological sample, and/or other suitable sample processing operations.

In variations of Block S110, lysing a biological sample and/or disrupting membranes in cells of a biological sample preferably includes physical methods (e.g., bead beating, nitrogen decompression, homogenization, sonication), which omit certain reagents that produce bias in representation of certain bacterial groups upon sequencing. Additionally or alternatively, lysing or disrupting in Block S110 can involve chemical methods (e.g., using a detergent, using a solvent, using a surfactant, etc.). Additionally or alternatively, lysing or disrupting in Block S110 can involve biological methods. In variations, separation of undesired elements can include removal of RNA using RNases and/or removal of proteins using proteases. In variations, purification of nucleic acids can include one or more of: precipitation of nucleic acids from the biological samples (e.g., using alcohol-based precipitation methods), liquid-liquid based purification techniques (e.g., phenol-chloroform extraction), chromatography-based purification techniques (e.g., column adsorption), purification techniques involving use of binding moiety-bound particles (e.g., magnetic beads, buoyant beads, beads with size distributions, ultrasonically responsive beads, etc.) configured to bind nucleic acids and configured to release nucleic acids in the presence of an elution environment (e.g., having an elution solution, providing a pH shift, providing a temperature shift, etc.), and any other suitable purification techniques.

In variations of Block S110, amplification of purified nucleic acids preferably includes one or more of: polymerase chain reaction (PCR)-based techniques (e.g., solid-phase PCR, RT-PCR, qPCR, multiplex PCR, touchdown PCR, nanoPCR, nested PCR, hot start PCR, etc.), helicase-dependent amplification (HDA), loop mediated isothermal amplification (LAMP), self-sustained sequence replication (3SR), nucleic acid sequence based amplification (NASBA), strand displacement amplification (SDA), rolling circle amplification (RCA), ligase chain reaction (LCR), and any other suitable amplification technique. In amplification of purified nucleic acids, the primers used are preferably selected to prevent or minimize amplification bias, as well as configured to amplify nucleic acid regions/sequences (e.g., of the 16S rRNA region, the 18S rRNA region, the ITS region, etc.) that are informative taxonomically, phylogenetically, for diagnostics, for formulations (e.g., for probiotic formulations), and/or for any other suitable purpose. Thus, universal primers (e.g., a F27-R338 primer set for 16S rRNA, a F515-R806 primer set for 16S rRNA, etc.) configured to avoid amplification bias can be used in amplification. Primers used in variations of Block S110 can additionally or alternatively include incorporated barcode sequences specific to each biological sample, which can facilitate identification of biological samples post-amplification. Primers used in variations of Block S110 can additionally or alternatively include adaptor regions configured to cooperate with sequencing techniques involving complementary adaptors (e.g., Illumina Sequencing). Additionally or alternatively, Block S110 can implement any other step configured to facilitate processing (e.g., using a Nextera kit).

In variations of Block S110, sequencing of purified nucleic acids can include methods involving targeted amplicon sequencing and/or metagenomic sequencing, implementing techniques including one or more of: sequencing-by-synthesis techniques (e.g., Illumina sequencing), capillary sequencing techniques (e.g., Sanger sequencing), pyrosequencing techniques, a nanopore sequencing techniques (e.g., using an Oxford Nanopore technique), or any other suitable sequencing technique.

In a specific example of Block S110, amplification and sequencing of nucleic acids from biological samples of the set of biological samples includes: solid-phase PCR involving bridge amplification of DNA fragments of the biological samples on a substrate with oligo adapters, where amplification involves primers having a forward index sequence (e.g., corresponding to an Illumina forward index for MiSeq/NextSeq/HiSeq platforms), a forward barcode sequence, a transposase sequence (e.g., corresponding to a transposase binding site for MiSeq/NextSeq/HiSeq platforms), a linker (e.g., a zero, one, or two-base fragment configured to reduce homogeneity and improve sequence results), an additional random base, a sequence for targeting a specific target region (e.g., a 16S rRNA region, a 18S rRNA region, a ITS region), a reverse index sequence (e.g., corresponding to an Illumina reverse index for MiSeq/HiSeq platforms), and a reverse barcode sequence. In the specific example, sequencing includes Illumina sequencing (e.g., with a HiSeq platform, with a MiSeq platform, with a NextSeq platform, etc.) using a sequencing-by-synthesis technique.

Some variations of sample processing in Block S110 can include further purification of amplified nucleic acids (e.g., PCR products) prior to sequencing, which functions to remove excess amplification elements (e.g., primers, dNTPs, enzymes, salts, etc.). In examples, additional purification can be facilitated using any one or more of: purification kits, buffers, alcohols, pH indicators, chaotropic salts, nucleic acid binding filters, centrifugation, and any other suitable purification technique.

In variations, computational processing in Block S110 can include any one or more of: identification of microbiome-derived sequences (e.g., as opposed to subject sequences and contaminants), alignment and mapping of microbiome-derived sequences (e.g., alignment of fragmented sequences using one or more of single-ended alignment, ungapped alignment, gapped alignment, pairing), and generating features derived from compositional and/or functional aspects of the microbiome associated with a biological sample.

In Block S110, identification of microbiome-derived sequences can include mapping of sequence data from sample processing to a subject reference genome (e.g., provided by the Genome Reference Consortium), in order to remove subject genome-derived sequences. Unidentified sequences remaining after mapping of sequence data to the subject reference genome can then be further clustered into operational taxonomic units (OTUs) based upon sequence similarity and/or reference-based approaches (e.g., using VAMPS, using MG-RAST, using QIIME databases), aligned (e.g., using a genome hashing approach, using a Needleman-Wunsch algorithm, using a Smith-Waterman algorithm), and mapped to reference bacterial genomes (e.g., provided by the National Center for Biotechnology Information), using an alignment algorithm (e.g., Basic Local Alignment Search Tool, FPGA accelerated alignment tool, BWT-indexing with BWA, BWT-indexing with SOAP, BWT-indexing with Bowtie, etc.). Mapping of unidentified sequences can additionally or alternatively include mapping to reference archaeal genomes, viral genomes and/or eukaryotic genomes. Furthermore, mapping of taxa can be performed in relation to existing databases, and/or in relation to custom-generated databases. In an example, Block S110 can include determining alignments between microorganism nucleic acid sequences and reference sequences associated with the C. difficile condition, where generating the microbiome composition dataset and the microbiome functional diversity dataset is based on the alignments.

In Block S110, upon identification of represented groups of microorganisms of the microbiome associated with a biological sample, generating features derived from compositional and functional aspects of the microbiome associated with a biological sample can be performed. Additionally or alternatively, generated features can include generating features that describe the presence or absence of certain taxonomic groups of microorganisms, and/or ratios between exhibited taxonomic groups of microorganisms. Additionally or alternatively, generating features can include generating features describing one or more of: quantities of represented taxonomic groups, networks of represented taxonomic groups, correlations in representation of different taxonomic groups, interactions between different taxonomic groups, products produced by different taxonomic groups, interactions between products produced by different taxonomic groups, ratios between dead and alive microorganisms (e.g., for different represented taxonomic groups, based upon analysis of RNAs), phylogenetic distance (e.g., in terms of Kantorovich-Rubinstein distances, Wasserstein distances etc.), any other suitable taxonomic group-related feature(s), any other suitable genetic or functional feature(s).

In relation to Block S110, additionally or alternatively, generating features can include generating features describing relative abundance of different microorganism groups, for instance, using a sparCC approach, using Genome Relative Abundance and Average size (GAAS) approach and/or using a Genome Relative Abundance using Mixture Model theory (GRAMMy) approach that uses sequence-similarity data to perform a maximum likelihood estimation of the relative abundance of one or more groups of microorganisms. Additionally or alternatively, generating features can include generating statistical measures of taxonomic variation, as derived from abundance metrics. Additionally or alternatively, generating features can include generating features derived from relative abundance factors (e.g., in relation to changes in abundance of a taxon, which affects abundance of other taxa). Additionally or alternatively, generating features can include generation of qualitative features describing presence of one or more taxonomic groups, in isolation and/or in combination. Additionally or alternatively, generating features can include generation of features related to genetic markers (e.g., representative 16S rRNA, 18S rRNA, and/or ITS sequences) characterizing microorganisms of the microbiome associated with a biological sample. Additionally or alternatively, generating features can include generation of features related to functional associations of specific genes and/or organisms having the specific genes. Additionally or alternatively, generating features can include generation of features related to pathogenicity of a taxon and/or products attributed to a taxon. Block S120 can, however, include generation of any other suitable feature(s) derived from sequencing and mapping of nucleic acids of a biological sample. For instance, the feature(s) can be combinatory (e.g. involving pairs, triplets), correlative (e.g., related to correlations between different features), and/or related to changes in features (e.g., temporal changes, changes across sample sites, spatial changes, etc.). However, Block S110 can be performed in any suitable manner, some embodiments, variations, and examples of which are described in U.S. application Ser. No. 14/593,424 filed 9 Jan. 2015, U.S. application Ser. No. 15/198,818 filed 30 Jun. 2016, U.S. application Ser. No. 15/098,027 filed 13 Apr. 2016, U.S. application Ser. No. 15/098,248 filed 13 Apr. 2016, U.S. application Ser. No. 15/098,236 filed 13 Apr. 2016, Ser. No. 15/098,222 filed 13 Apr. 2016, U.S. application Ser. No. 15/098,204 filed 13 Apr. 2016, U.S. application Ser. No. 15/098,174 filed 13 Apr. 2016, U.S. application Ser. No. 15/098,110 filed 13 Apr. 2016, U.S. application Ser. No. 15/098,081 filed 13 Apr. 2016, U.S. application Ser. No. 15/098,153 filed 13 Apr. 2016, U.S. application Ser. No. 15/228,890 filed 4 Aug. 2016, and U.S. application Ser. No. 15/240,919 filed 18 Aug. 2016, which are each hereby incorporated in their entirety by this reference.

Block S120 recites: receiving a supplementary dataset informative of the Clostridium-associated condition for at least a subset of the population of subjects. Block S120 functions to acquire additional data associated with one or more subjects of the set of subjects, which can be used to train and/or validate the characterization process generated in Block S130. In Block S120, the supplementary dataset preferably includes survey-derived data, but can additionally or alternatively include any one or more of: contextual data derived from sensors, medical data (e.g., current and historical medical data), and any other suitable type of data. In variations of Block S120 including reception of survey-derived data, the survey-derived data preferably provides physiological, demographic, and behavioral information in association with a subject. Physiological information can include information related to physiological features (e.g., height, weight, body mass index, body fat percent, body hair level, etc.). Demographic information can include information related to demographic features (e.g., gender, age, ethnicity, marital status, number of siblings, socioeconomic status, sexual orientation, etc.). Behavioral information can include information related to one or more of: health conditions (e.g., health and disease states), living situations (e.g., living alone, living with pets, living with a significant other, living with children, etc.), dietary habits (e.g., omnivorous, vegetarian, vegan, sugar consumption, acid consumption, etc.), behavioral tendencies (e.g., levels of physical activity, drug use, alcohol use, etc.), different levels of mobility (e.g., related to distance traveled within a given time period), different levels of sexual activity (e.g., related to numbers of partners and sexual orientation), and any other suitable behavioral information. Survey-derived data can include quantitative data, qualitative data that can be converted to quantitative data (e.g., using scales of severity, mapping of qualitative responses to quantified scores, etc.), and/or other suitable data.

In facilitating reception of survey-derived data, Block S120 can include providing one or more surveys to a subject of the population of subjects, or to an entity associated with a subject of the population of subjects. Surveys can be provided in person (e.g., in coordination with sample provision and reception from a subject), electronically (e.g., during account setup by a subject, at an application executing at an electronic device of a subject, at a web application accessible through an internet connection, etc.), and/or in any other suitable manner.

For Block S120, additionally or alternatively, portions of the supplementary dataset can be derived from sensors associated with the subject(s) (e.g., sensors of wearable computing devices, sensors of mobile devices, biometric sensors associated with the user, etc.). As such, Block S130 can include receiving one or more of: physical activity- or physical action-related data (e.g., accelerometer and gyroscope data from a mobile device or wearable electronic device of a subject), environmental data (e.g., temperature data, elevation data, climate data, light parameter data, etc.), patient nutrition or diet-related data (e.g., data from food establishment check-ins, data from spectrophotometric analysis, etc.), biometric data (e.g., data recorded through sensors within the patient's mobile computing device, data recorded through a wearable or other peripheral device in communication with the patient's mobile computing device), location data (e.g., using GPS elements), and any other suitable data. Additionally or alternatively, portions of the supplementary dataset can be derived from medical record data and/or clinical data of the subject(s). As such, portions of the supplementary dataset can be derived from one or more electronic health records (EHRs) of the subject(s). Additionally or alternatively, the supplementary dataset of Block S120 can include any other suitable diagnostic information (e.g., clinical diagnosis information), which can be combined with analyses derived from features to support characterization of subjects in subsequent blocks of the method 100. For instance, information derived from a colonoscopy, biopsy, blood test, diagnostic imaging, survey-related information, and any other suitable test can be used to supplement Block S120. However, supplementary datasets and receiving supplementary datasets can be configured in any suitable manner.

Block S130 recites: performing a characterization process derived from the supplementary dataset and features extracted from at least one of the microbiome composition dataset and microbiome functional diversity dataset. Block S130 functions to identify features and/or feature combinations that can be used to characterize subjects or groups based upon their microbiome composition and/or functional features. Block S130 can additionally or alternatively function to generate a characterization model (e.g., using identified features) for determining characterizations associated with a Clostridium-associated condition. As such, the characterization process can be used as a diagnostic tool that can characterize a subject (e.g., in terms of behavioral traits, in terms of medical conditions, in terms of demographic traits, etc.) based upon their microbiome composition and/or functional features, in relation to one or more of their health condition states, behavioral traits, medical conditions, demographic traits, and any other suitable traits. Such characterization can then be used to suggest or provide personalized therapies by way of the therapy model of Block S140.

In performing the characterization process, Block S130 can use computational methods (e.g., statistical methods, machine learning methods, artificial intelligence methods, bioinformatics methods, etc.) to characterize a subject as exhibiting features characteristic of a group of subjects with a health condition.

In one variation of Block S130, characterization can be based upon features determined in accordance with feature-selection rules (e.g., Clostridium-associated condition feature-selection rules defining correlations between microbiome features and one or more Clostridium-associated conditions). For example, Block S130 can include obtaining a set of Clostridium (e.g., C. difficile) associated feature-selection rules correlating the Clostridium-associated condition to a subset of microbiome composition features and a subset of microbiome functional diversity features; and generating a feature set based on evaluating the microbiome composition dataset and the microbiome functional diversity dataset against the set of Clostridium feature-selection rules. The feature-selection rules can include one or more of: application of statistical analysis operations (e.g., an analysis of probability distributions, etc.), supplementary dataset-based feature-selection rules (e.g., selecting features correlated with supplementary dataset informative of a Clostridium-associated condition, etc.), processing-based feature-selection rules (e.g., selecting amount and/or type of features based on processing efficiency and/or other processing constraints, etc.), accuracy-based feature-selection rules (e.g., filtering irrelevant and/or redundant features in relation to the Clostridium-associated condition, etc.), user-selected feature-selection rules, and/or any other suitable feature-selection rules. For example, feature-selection rules can include application of a statistical analysis of similarities and/or differences between a first group of subjects exhibiting a target state (e.g., a health condition state) and a second group of subjects not exhibiting the target state (e.g., a “normal” state). In implementing this variation, one or more of a Kolmogorov-Smirnov (KS) test, a permutation test, a Cramér-von Mises test, and any other statistical test (e.g., t-test, Welch's t-test, z-test, chi-squared test, test associated with distributions, etc.) can be used. In particular, one or more such statistical hypothesis tests can be used to assess a set of features having varying degrees of abundance in a first group of subjects exhibiting a target state (e.g., a sick state) and a second group of subjects not exhibiting the target state (e.g., having a normal state). In more detail, the set of features assessed can be constrained based upon percent abundance and/or any other suitable parameter pertaining to diversity in association with the first group of subjects and the second group of subjects, in order to increase or decrease confidence in the characterization. In a specific implementation of this example, a feature can be derived from a taxon of bacteria that is abundant in a certain percentage of subjects of the first group and subjects of the second group, where a relative abundance of the taxon between the first group of subjects and the second group of subjects can be determined from the KS test, with an indication of significance (e.g., in terms of p-value). Thus, an output of Block S130 can include a normalized relative abundance value (e.g., 25% greater abundance of a taxon in sick subjects vs. healthy subjects) with an indication of significance (e.g., a p-value of 0.0013). Variations of feature generation can additionally or alternatively implement or be derived from functional features or metadata features (e.g., non-bacterial markers). Different feature-selection rules can be customized (e.g., in generating a model) for different demographic groups, subjects, types of supplementary data, and/or other suitable criteria. For example, Block S130 can include applying a first set of feature-selection rules to define a first feature subset for generating a first characterization model of a first C. difficile strain, and applying a second set of feature-selection rules to define a second feature subset for generating a first characterization model of a second C. difficile strain. However, any suitable number and/or type of feature-selection rules can be applied in any manner to define one or more feature sets.

In performing the characterization process, Block S130 can additionally or alternatively transform input data from at least one of the microbiome composition dataset and microbiome functional diversity dataset into feature vectors that can be tested for efficacy in predicting characterizations of the population of subjects. For example, Block S130 can include generating a set of microbiome feature vectors for a set of users (e.g., a population of subjects) based on a subset of microbiome composition features and a subset of microbiome functional diversity features, and training a characterization model with the set of microbiome feature vectors. Data from the supplementary dataset can be used to provide indication of one or more characterizations of a set of characterizations, where the characterization process is trained with a training dataset of candidate features and candidate classifications to identify features and/or feature combinations that have high degrees (or low degrees) of predictive power in accurately predicting a classification. As such, refinement of the characterization process with the training dataset identifies feature sets (e.g., of subject features, of combinations of features) having high correlation with specific classifications of subjects.

In variations of Block S130, feature vectors effective in predicting classifications of the characterization process can include features related to one or more of: microbiome diversity metrics (e.g., in relation to distribution across taxonomic groups, in relation to distribution across archaeal, bacterial, viral, and/or eukaryotic groups), presence of taxonomic groups in one's microbiome, representation of specific genetic sequences (e.g., 16S rRNA sequences) in one's microbiome, relative abundance of taxonomic groups in one's microbiome, microbiome resilience metrics (e.g., in response to a perturbation determined from the supplementary dataset), abundance of genes that encode proteins or RNAs with given functions (enzymes, transporters, proteins from the immune system, hormones, interference RNAs, etc.) and any other suitable features derived from the microbiome diversity dataset, the microbiome functional diversity dataset, and/or the supplementary dataset. For example, features can include a functional diversity feature associated with bile acid metabolism, and/or a composition feature associated with a relative abundance of Bacteroidetes, Firmicutes, and Proteobacteria, where the features can be used in generating and/or applying a characterization model (e.g., for characterizing a C. difficile Ribotype 027 strain infection including at least one of sepsis and colitis, etc.). Additionally, combinations of features can be used in a feature vector, where features can be grouped and/or weighted in providing a combined feature as part of a feature set. For example, one feature or feature set can include a weighted composite of the number of represented classes of bacteria in one's microbiome, presence of a specific genus of bacteria in one's microbiome, representation of a specific 16S rRNA sequence in one's microbiome, and relative abundance of a first phylum over a second phylum of bacteria. However, the feature vectors can additionally or alternatively be determined in any other suitable manner.

In a variation, Block S130 can include generating a characterization model based on one or more features (e.g., described above) and/or supplementary data, but characterization models can be generated based on any suitable data. For example, Block S130 can include applying a feature set (e.g., generated based on Clostridium-associated condition feature-selection rules) with the supplementary dataset to generate a characterization model for the C. difficile-associated condition. Different characterization models can be generated for different demographic groups (e.g., a first characterization model characterizing a Clostridium-associated condition for recently released hospital patients, a second characterization model for antibiotic users, etc.), individual subjects, supplementary data (e.g., models incorporating features derived from biometric sensor data vs. models independent of supplementary data, etc.), and/or other suitable criteria. In an example, the method can include generating a characterization model for a demographic group of exercisers; associating the characterization model with user accounts (e.g., at a database of the processing system) for subjects who indicate physical activity (e.g., at a digital survey presented by the interface); and retrieving the characterization model (e.g., from the database) for characterizing the subjects. Generating a plurality of characterization models suited to different contexts can confer improvements to the processing system by improving characterization accuracy (e.g., by tailoring analysis to a particular subject's demographic and/or situation, etc.), retrieval of the appropriate characterization model from a database (e.g., by associating customized characterization models with particular user accounts and/or other identifiers), training and/or execution of characterization models (e.g., when the customized models are associated with a subset of a pool of potential Clostridium-associated condition features, where the remaining features are less pertinent to a particular subject), and/or other suitable aspects of the processing system.

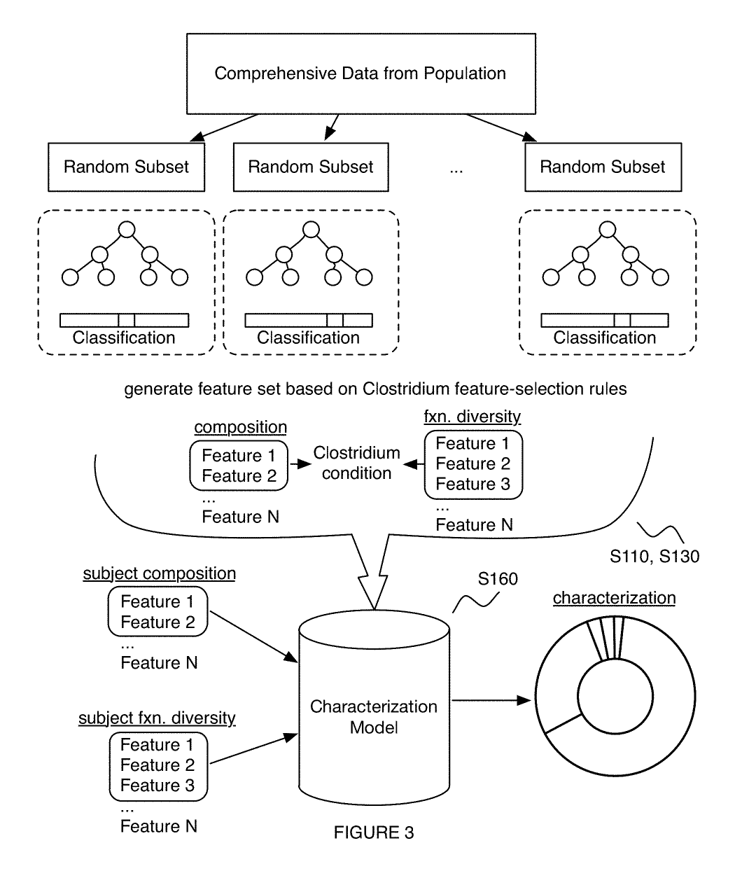

In Block S130, as shown in FIG. 3, in an example of the variation of Block S130, the characterization process can be generated and trained according to a random forest predictor (RFP) algorithm that combines bagging (e.g., bootstrap aggregation) and selection of random sets of features from a training dataset to construct a set of decision trees, T, associated with the random sets of features. In using a random forest algorithm, N cases from the set of decision trees are sampled at random with replacement to create a subset of decision trees, and for each node, m prediction features are selected from all of the prediction features for assessment. The prediction feature that provides the best split at the node (e.g., according to an objective function) is used to perform the split (e.g., as a bifurcation at the node, as a trifurcation at the node). By sampling many times from a large dataset, the strength of the characterization process, in identifying features that are strong in predicting classifications can be increased substantially. In this variation, measures to prevent bias (e.g., sampling bias) and/or account for an amount of bias can be included during processing to increase robustness of the model. Additionally or alternatively, any number of characterization models can be generated for any suitable purpose. However, performing a characterization process can be performed in any suitable manner.

4.1 Method—C. difficile Characterization

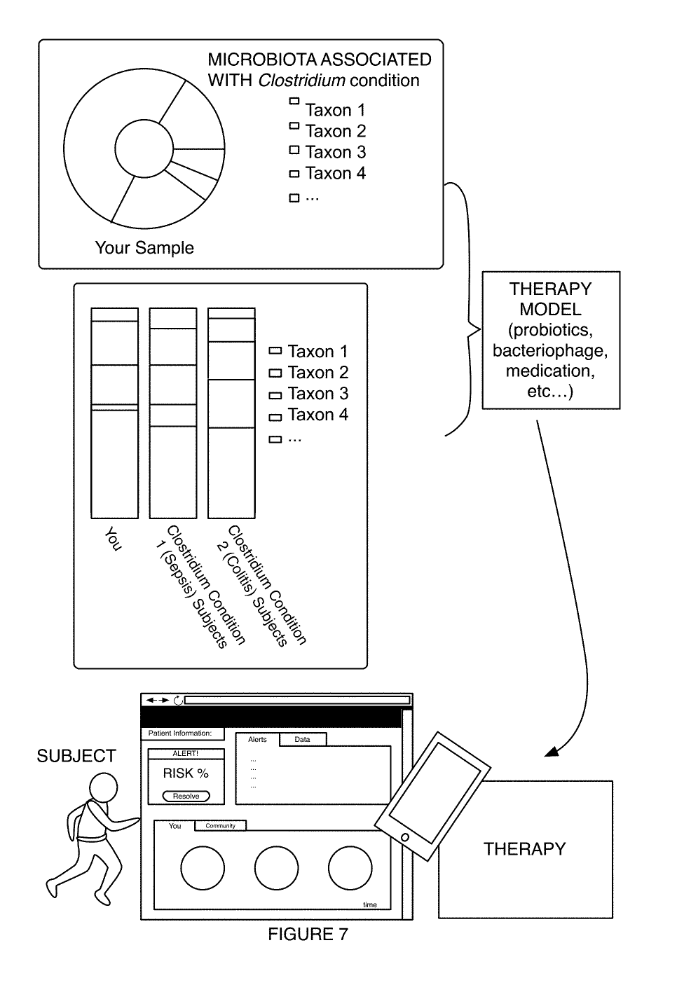

In one implementation, a characterization process of Block S130 based upon statistical analyses can identify the sets of features that have the highest correlations with a C. difficile-associated condition. In some applications, as shown in FIG. 7, the characterization process of Block S130 can facilitate identification of which microorganism population(s) are upregulated or downregulated in relation to C. difficile activity, and/or which microbiome functional aspects (e.g., in relation to COG/KEGG pathways) are upregulated or downregulated in relation to C. difficile activity. In an example, composition and/or functional diversity related to C. difficile can be characterized in relation to other species within the Clostridium genus.

Furthermore, as shown in FIG. 8, the characterization processes of Block S130 can include 1) characterizing strains of C. difficile (e.g., Ribotype 027, Ribotype 002, Ribotype 106, Ribotype 017, Ribotype 078, etc.) present in a sample, and 2) characterizing, at the strain level, relationship(s) between C. difficile strains and microorganism population and/or functional aspect upregulation/downregulation (e.g., upregulation/downregulation of particular C. difficile strains in relation to upregulation/downregulation of other taxonomic groups and/or other strains). In a specific example, the method 100 can be used to identify 98% of all C. difficile strains present in a sample from the subject(s), as well as relationships between the strain(s) present and activity (e.g., in relation to upregulation or downregulation, in relation to functional activity) of other microorganisms associated with the sample from the subject(s). In the specific example, the method 100 can identify the strains(s) of C. difficile present in a sample, relationships between the strain(s) and Bifidobacterial populations of the sample, and functional aspects in relation to pH and/or butyrate modulation. In another example, the method 100 can include identifying between specific C. difficile strains and microbiome composition features, functional diversity, and/or Clostridium-associated conditions. However, characterizing aspects in relation to Clostridium strains can be performed in any suitable manner.

In a variation of the characterization process of Block S130, a set of features useful for characterizations associated with C. difficile includes features derived from one or more of the following taxa: Flavonifractor plautii (species), Bifidobacterium longum (species), Bacteroides fragilis (species), Bifidobacterium bifidum (species), Erysipelatoclostridium ramosum (species), Parabacteroides distasonis (species), Bacteroides vulgatus (species), Faecalibacterium prausnitzii (species), Blautia sp. YHC-4 (species), Blautia faecis (species), Bacteroides acidifaciens (species), Collinsella aerofaciens (species), Anaerostipes caccae (species), bacterium NLAE-zl-P855 (species), Bacteroides thetaiotaomicron (species), Bacteroides vulgatus (species), Bacteroides xylanisolvens (species), Bilophila wadsworthia (species), Blautia product (species), Clostridium clostridioforme (species), Clostridium hathewayi (species), Clostridium innocuum (species), Clostridium symbiosum (species), Eggerthella lenta (species), Escherichia coli (species), Haemophilus parainfluenzae (species), Intestinibacter bartlettii (species), Ruminococcus gnavus (species), Ruminococcus torques (species), Roseburia (genus), Veillonella (genus), Kluyvera (genus), Sarcina (genus), Subdoligranulum (genus), Bifidobacterium (genus), Faecalibacterium (genus), Bilophila (genus), Lactobacillus (genus), Eubacterium (genus), Parabacteroides (genus), Akkermansia (genus), Dorea (genus), Bacteroides (genus), Moryella (genus), Anaerotruncus (genus), Enterococcus (genus), Eggerthella (genus), Collinsella (genus), Anaerobacter (genus), Megasphaera (genus), Alistipes (genus), Intestinimonas (genus), Streptococcus (genus), Anaerostipes (genus), Blautia (genus), Escherichia-Shigella (genus), Haemophilus (genus), Hungatella (genus), Intestinibacter (genus), Lachnoclostridium (genus), Flavonifractor (genus), Clostridium (genus), Peptoclostridium (genus), Pseudobutyrivibrio (genus), Erysipelatoclostridium (genus), Ruminococcaceae (family), Enterobacteriaceae (family), Coriobacteriaceae (family), Lactobacillaceae (family), Lachnospiraceae (family), Bifidobacteriaceae (family), Eubacteriaceae (family), Verrucomicrobiaceae (family), Bacteroidaceae (family), Oscillospiraceae (family), Enterococcaceae (family), Rikenellaceae (family), Bradyrhizobiaceae (family), Clostridiaceae (family), Peptostreptococcaceae (family), Veillonellaceae (family), Christensenellaceae (family), Erysipelotrichaceae (family), Streptococcaceae (family), Enterobacteriales (order), Clostridiales (order), Coriobacteriales (order), Bifidobacteriales (order), Verrucomicrobiales (order), Selenomonadales (order), Erysipelotrichales (order), Lactobacillales (order), Clostridia (class), Actinobacteria (class), Verrucomicrobiae (class), Alphaproteobacteria (class), Deltaproteobacteria (class), Negativicutes (class), Erysipelotrichia (class), Gammaproteobacteria (class), Bacilli (class), Proteobacteria (phylum), Actinobacteria (phylum), Verrucomicrobia (phylum) and Firmicutes (phylum).

Additionally or alternatively, in Block S130, the set of features associated with a C. difficile-related condition can be derived from one or more of: a clusters of orthologous groups (COG) code, a cellular processes and signaling Kyoto Encyclopedia of Genes and Genomes (KEGG) pathway derived feature, a metabolism KEGG pathway derived feature, a signaling molecules and interaction KEGG pathway derived feature, a translation KEGG pathway derived feature, an other ion-coupled transporters KEGG pathway derived feature, a bacterial toxins KEGG pathway derived feature, a caprolactam degradation KEGG pathway derived feature, an ascorbate and aldarate metabolism KEGG pathway derived feature, an inorganic ion transport and metabolism KEGG pathway derived feature, a protein SCO1/2 KEGG pathway derived feature (e.g., a K07152 KEGG code associated with protein SCO1/2), a cytochrome KEGG pathway derived feature (e.g., a K00413 KEGG code associated with CYC1, CYT1, petC—ubiquinol-cytochrome c reductase cytochrome ci subunit), a nitrogen regulation KEGG pathway derived feature (e.g., a K13599 KEGG code associated with two-component system, NtrC family, nitrogen regulation response regulator NtrX), an oxidoreductase acting on paired donors, with incorporation or reduction of molecular oxygen KEGG pathway derived feature (e.g., a K00517 KEGG code associated with bisphenol degradation, polycyclic aromatic hydrocarbon degradation, aminobenzoate degradation, limonene and pinene degradation, stilbenoid, diarylheptanoid and gingerol biosynthesis), a putative membrane protein KEGG pathway derived feature (e.g., a K08973 KEGG code associated with putative membrane protein), a UQCRFS1/RIP1/petA KEGG pathway derived feature (e.g., a K00412 KEGG code associated with ubiquinol-cytochrome c reductase iron-sulfur subunit [EC 1.10.2.2]), a CYTB/petB KEGG pathway derived feature (e.g., a KEGG K00412 code associated with CYTB, petB, ubiquinol-cytochrome c reductase cytochrome b subunit), a cobS KEGG pathway derived feature (e.g., a KEGG K09882 code associated with cobaltochelatase CobS [EC 6.6.1.2]), and a K07018 KEGG pathway derived feature (e.g., a feature associated with an uncharacterized protein). In an example, the features can include KEGG functional features associated with at least one of: pentose phosphate pathway, gluconeogenesis, and carbon fixation.

Thus, characterization of the subject in Block S130 can include characterization of the subject in relation to a C. difficile-based health condition, based upon detection of one or more of the above features, in a manner that is an alternative or supplemental to typical methods of diagnosis or characterization. In variations of the specific example, the set of features can, however, include any other suitable features useful for diagnostics/characterization of a subject. Characterization of the subject(s) in Block S130 can additionally or alternatively implement use of a high false positive test and/or a high false negative test to further analyze sensitivity of the characterization process in supporting analyses generated according to embodiments of the method 100.

In another variation, characterizing a Clostridium-associated condition in Block S130 can include generating a diagnostic analysis of a Clostridium infection (e.g., estimating a risk of infection, diagnosing the infection, etc.) and/or associated complications including any one or more of: a C. difficile infection (e.g., sepsis, colitis, toxic megacolon, colon perforation, anaerobic infection, etc.), Clostridium Botulinum infection (e.g., botulism, flaccid paralytic disease, etc.), Clostridium perfringens infection (e.g., cellulitis, fascitis, gas gangrene, tissue necrosis, bacteremia, emphysematous cholecystitis, etc.), Clostridium tetani (e.g., tetanus, etc.), and/or any other suitable infections and/or complications. Generating a diagnostic analysis can be based on relative abundance of taxonomic groups (e.g., diagnosing a C. difficile infection based on a high abundance of C. difficile Ribotype 027 strain; estimating an increased infection risk based on a decreased abundance of a taxonomic group correlated with defending against a Clostridium infection), functional diversity (e.g., based on bile acid metabolism; estimating a decreased infection risk based on increased production of types of bile acids such as Chenodeoxycholic acid inhibiting Clostridium spore germination; etc.), and/or any other suitable data.

In another variation of Block S130, characterizing a Clostridium-associated condition can be based on one or more supplementary datasets. For example, the set of Clostridium-associated feature-selection rules can correlate the Clostridium infection to a biometric feature derived from biometric sensor data informative of a Clostridium-associated condition (e.g., temperature data, cardiovascular data, blood data, stool data, etc. indicating the presence of symptoms such as fever, nausea, abdominal pain, diarrhea, etc.). In another example, performing a characterization process (e.g., generating a characterization model) can be based on antibiotic and/or probiotic regimen data associated with a population of users, where particular regimens can aid in illuminating microbiome compositions and/or functional diversity correlated with Clostridium-associated conditions. However, performing a characterization process in relation to a Clostridium-associated condition can be performed in any suitable manner.

### 4.2 Method—Therapy

The method 100 can additionally or alternatively include Block S140, which recites: determining a therapy for preventing, ameliorating, and/or otherwise modifying a Clostridium-associated condition. Block S140 functions to identify and/or predict therapies (e.g., probiotic-based therapies, phage-based therapies, small molecule-based therapies, fecal matter transplant-based therapies, etc.) that can shift a subject's microbiome composition and/or functional features toward a desired equilibrium state in promotion of the subject's health (e.g., reduce the risk of a Clostridium-associated condition, ameliorate a Clostridium-associated condition, etc.). Block S140 can additionally or alternatively include generating and/or applying a therapy model for determining the therapy.

In Block S140, the therapies can be selected from therapies including one or more of: probiotic therapies, phage-based therapies, small molecule-based therapies, fecal matter transplant-based therapies, cognitive/behavioral therapies, physical rehabilitation therapies, clinical therapies, medication-based therapies, diet-related therapies, and/or any other suitable therapy designed to operate in any other suitable manner in promoting a user's health. In a specific example of a bacteriophage-based therapy, one or more populations (e.g., in terms of colony forming units) of phages specific to a certain bacteria (or other microorganism) represented in the subject can be used to down-regulate or otherwise eliminate populations of the certain bacteria. As such, bacteriophage-based therapies can be used to reduce the size(s) of the undesired population(s) of bacteria represented in the subject. Complementarily, bacteriophage-based therapies can be used to increase the relative abundances of bacterial populations not targeted by the bacteriophage(s) used.

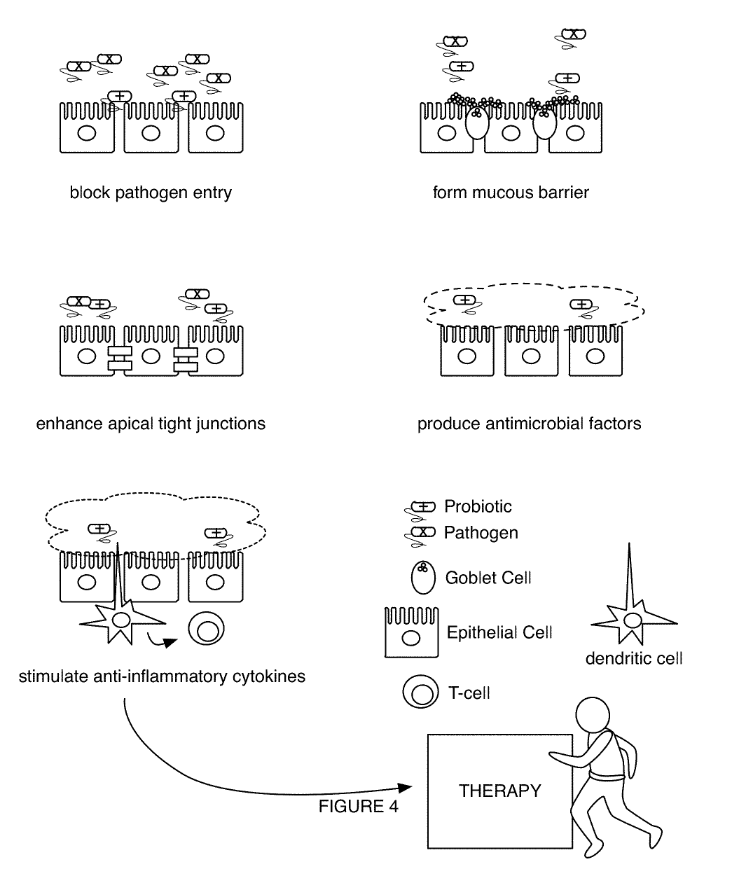

Relating to Block S140, in another specific example of probiotic therapies, as shown in FIG. 4, candidate therapies of the therapy model can perform one or more of: blocking pathogen entry into an epithelial cell by providing a physical barrier (e.g., by way of colonization resistance), inducing formation of a mucous barrier by stimulation of goblet cells, enhance integrity of apical tight junctions between epithelial cells of a subject (e.g., by stimulating up regulation of zona-occludens 1, by preventing tight junction protein redistribution), producing antimicrobial factors, stimulating production of anti-inflammatory cytokines (e.g., by signaling of dendritic cells and induction of regulatory T-cells), triggering an immune response, and performing any other suitable function that adjusts a subject's microbiome away from a state of dysbiosis.

In variations of Block S140, the therapy model is preferably based upon data from a large population of subjects, which can include the population of subjects from which the microbiome diversity datasets are derived in Block S110, where microbiome composition and/or functional features or states of health, prior exposure to and post exposure to a variety of therapeutic measures, are well characterized. Such data can be used to train and validate the therapy provision model, in identifying therapeutic measures that provide desired outcomes for subjects based upon different microbiome characterizations. In variations, support vector machines, as a supervised machine learning algorithm, can be used to generate the therapy provision model. However, any other suitable machine learning algorithm described above can facilitate generation of the therapy provision model. Processing of therapy models can be analogous to processing of characterization models (e.g., described for Block S130), where any number of treatment models can be generated for different purposes (e.g., different demographic groups, individuals, supplementary datasets, etc.), associated with user accounts and/or other identifiers, and/or otherwise processed for customizing therapy determination and/or promotion for different subjects.

Regarding Block S140, while some methods of statistical analyses and machine learning are described in relation to performance of the Blocks above, variations of the method 100 can additionally or alternatively utilize any other suitable algorithms in performing the characterization process. In variations, the algorithm(s) can be characterized by a learning style including any one or more of: supervised learning (e.g., using logistic regression, using back propagation neural networks), unsupervised learning (e.g., using an Apriori algorithm, using K-means clustering), semi-supervised learning, reinforcement learning (e.g., using a Q-learning algorithm, using temporal difference learning), and any other suitable learning style. Furthermore, the algorithm(s) can implement any one or more of: a regression algorithm (e.g., ordinary least squares, logistic regression, stepwise regression, multivariate adaptive regression splines, locally estimated scatterplot smoothing, etc.), an instance-based method (e.g., k-nearest neighbor, learning vector quantization, self-organizing map, etc.), a regularization method (e.g., ridge regression, least absolute shrinkage and selection operator, elastic net, etc.), a decision tree learning method (e.g., classification and regression tree, iterative dichotomiser 3, C4.5, chi-squared automatic interaction detection, decision stump, random forest, multivariate adaptive regression splines, gradient boosting machines, etc.), a Bayesian method (e.g., naive Bayes, averaged one-dependence estimators, Bayesian belief network, etc.), a kernel method (e.g., a support vector machine, a radial basis function, a linear discriminant analysis, etc.), a clustering method (e.g., k-means clustering, expectation maximization, etc.), an associated rule learning algorithm (e.g., an Apriori algorithm, an Eclat algorithm, etc.), an artificial neural network model (e.g., a Perceptron method, a back-propagation method, a Hopfield network method, a self-organizing map method, a learning vector quantization method, etc.), a deep learning algorithm (e.g., a restricted Boltzmann machine, a deep belief network method, a convolutional network method, a stacked auto-encoder method, etc.), a dimensionality reduction method (e.g., principal component analysis, partial least squares regression, Sammon mapping, multidimensional scaling, projection pursuit, etc.), an ensemble method (e.g., boosting, bootstrapped aggregation, AdaBoost, stacked generalization, gradient boosting machine method, random forest method, etc.), and any suitable form of algorithm.

Additionally or alternatively, in Block S140, the therapy model can be derived in relation to identification of a “normal” or baseline microbiome composition and/or functional features, as assessed from subjects of a population of subjects who are identified to be in good health. Upon identification of a subset of subjects of the population of subjects who are characterized to be in good health (e.g., using features of the characterization process), therapies that modulate microbiome compositions and/or functional features toward those of subjects in good health can be generated in Block S140. Block S140 can thus include identification of one or more baseline microbiome compositions and/or functional features (e.g., one baseline microbiome for each of a set of demographics), and potential therapy formulations and therapy regimens that can shift microbiomes of subjects who are in a state of dysbiosis toward one of the identified baseline microbiome compositions and/or functional features. The therapy model can, however, be generated and/or refined in any other suitable manner.

Regarding Block S140, microorganism compositions associated with probiotic therapies associated with the therapy model preferably include microorganisms that are culturable (e.g., able to be expanded to provide a scalable therapy) and non-lethal (e.g., non-lethal in their desired therapeutic dosages). Furthermore, microorganism compositions can include a single type of microorganism that has an acute or moderated effect upon a subject's microbiome. Additionally or alternatively, microorganism compositions can include balanced combinations of multiple types of microorganisms that are configured to cooperate with each other in driving a subject's microbiome toward a desired state. For instance, a combination of multiple types of bacteria in a probiotic therapy can include a first bacteria type that generates products that are used by a second bacteria type that has a strong effect in positively affecting a subject's microbiome. Additionally or alternatively, a combination of multiple types of bacteria in a probiotic therapy can include several bacteria types that produce proteins with the same functions that positively affect a subject's microbiome.

Regarding Block S140, probiotic compositions can be naturally or synthetically derived. For instance, in one application, a probiotic composition can be naturally derived from fecal matter or other biological matter (e.g., of one or more subjects having a baseline microbiome composition and/or functional features, as identified using the characterization process and the therapy model). Additionally or alternatively, probiotic compositions can be synthetically derived (e.g., derived using a benchtop method) based upon a baseline microbiome composition and/or functional features, as identified using the characterization process and the therapy model. In variations, microorganism agents that can be used in probiotic therapies can include one or more of: yeast (e.g., Saccharomyces boulardii), gram-negative bacteria (e.g., E. coli Nissle), gram-positive bacteria (e.g., Lactobacillus rhamnosus, Lactobacillus acidophilus, Lactobacillus casei, Lactobacillus helveticus, Lactobacillus plantarum, Lactobacillus fermentum, Lactobacillus salivarius, Lactobacillus delbrueckii (including subsp. bulgaricus), Lactobacillus johnsonii, Lactobacillus reuteri, Lactobacillus gasseri, Lactobacillus brevis (including subsp. coagulans), Bifidobacterium animalis (including subsp. lactis), Bifidobacterium longum (including subsp. infantis), Bifidobacterium bifidum, Bifidobacterium pseudolongum, Bifidobacterium thermophilum, Bifidobacterium breve, Streptococcus thermophilus, Bacillus cereus, Bacillus subtilis, Bacillus polyfermenticus, Bacillus clausii, Bacillus licheniformis, Bacillus coagulans, Bacillus pumilus, Brevibacillus brevis, Lactococcus lactis, Leuconostoc mesenteroides, Enterococcus faecium, Enterococcus faecalis, Enterococcus durans, Clostridium butyricum, Propionibacterium freudenreichii, Sporolactobacillus inulinus, Sporolactobacillus vineae, Faecalibacterium prausnitzii, Prevotella bryantii, Pediococcus acidilactici, Pediococcus pentosaceus, Akkermansia muciniphila, etc.), and/or any other suitable type of microorganism agent.

For Block S140, in examples of probiotic therapies, probiotic compositions can include components of one or more of the identified taxa of microorganisms (e.g., as described in Section 4.1 above, provided at dosages of 1 million to 10 billion CFUs, as determined from a therapy model that predicts positive adjustment of a subject's microbiome in response to the therapy. Additionally or alternatively, the therapy can include dosages of proteins resulting from functional presence in the microbiome compositions of subjects without a specific condition. In the examples, a subject can be instructed to ingest capsules including the probiotic formulation according to a regimen tailored to one or more of his/her: physiology (e.g., body mass index, weight, height), demographics (e.g., gender, age), severity of dysbiosis, sensitivity to medications, and any other suitable factor.

Furthermore, in relation to C. difficile characterization and/or C. difficile strain characterization, Block S140 can include determination, based upon one or more analyses, of therapies (e.g., probiotic therapies, bacteriophage-based therapies, antibiotic therapies, fecal matter transplant therapies, etc.) that can be used to positively modulate a subject's microbiome composition and/or functional aspects in relation to improving the subject's C. difficile-associated condition. In particular, Block S140 can include identifying, prescribing, and/or providing therapies for downregulation and/or entirely eliminating C. difficile populations in the subject, while not adversely affecting the microbiome of the subject in any other manner (e.g., in relation to microorganism populations, in relation to functional aspects, etc.). In an example, the therapy can include recommending and/or controlling a central venous catheter for administering medications (e.g., antibiotics, steroids, blood pressure support, etc.) and/or fluids for ameliorating symptoms of sepsis, but any suitable therapy can be promoted in relation to treating Clostridium-based sepsis. In another example, the therapy can include recommending and/or otherwise facilitating a medication regimen, surgical procedures (e.g., colectomy, etc.), and/or other suitable therapies for treating Clostridium-based colitis. In another example, the therapy can include an antibiotic and/or probiotic regimen to facilitate a microbiome composition suitable for defending against or preventing Clostridium infection, such as a microbiome composition including a smaller proportion of Bacteroidetes and Firmicutes, and a higher proportion of Proteobacteria (e.g., relative other users, relative other user groups, relative averages and/or other statistics, etc.). Additionally or alternatively, a therapy can be operable to facilitate any suitable relative abundance of particular taxonomic groups, and/or any suitable microbiome composition. In another example, a therapy can include scheduling an appointment with care provider (e.g., in response to a Clostridium infection risk exceeding a threshold, such as based on microbiome functional diversity and supplementary datasets indicating lifestyle choices correlated with increased risk; in response to diagnosing a Clostridium infection; etc.). However, Block S140 can be performed in any suitable manner.

### 4.3 Method—Personalization

The method can additionally or alternatively include Block S150, which recites: processing a biological sample from a subject, which functions to receive and process a biological sample to facilitate generation of a microbiome dataset for the subject that can be used to derive inputs for the characterization process. As such, receiving, processing, and analyzing the biological sample preferably facilitates generation of a microbiome dataset for the subject, which can be used to provide inputs for a characterization process. In Block S150, the biological sample is preferably generated from the subject and/or an environment of the subject in a non-invasive manner. In variations, non-invasive manners of sample reception can use any one or more of: a permeable substrate (e.g., a swab configured to wipe a region of a subject's body, toilet paper, a sponge, etc.), a non-permeable substrate (e.g., a slide, tape, etc.) a container (e.g., vial, tube, bag, etc.) configured to receive a sample from a region of a subject's body, and any other suitable sample-reception element. In a specific example, the biological sample can be collected from one or more of the subject's nose, skin, genitals, mouth, and gut in a non-invasive manner (e.g., using a swab and a vial). However, the biological sample can additionally or alternatively be received in a semi-invasive manner or an invasive manner. In variations, invasive manners of sample reception can use any one or more of: a needle, a syringe, a biopsy element, a lance, and any other suitable instrument for collection of a sample in a semi-invasive or invasive manner. In specific examples, samples can include blood samples, plasma/serum samples (e.g., to enable extraction of cell-free DNA), tissue samples, and/or any other suitable sample.

Regarding Block S150, in the above variations and examples, the biological sample can be taken from the body of the subject without facilitation by another entity (e.g., a caretaker associated with a subject, a healthcare professional, an automated or semi-automated sample collection apparatus, etc.), or can alternatively be taken from the body of the subject with the assistance of another entity. In one example, where the biological sample is taken from the subject without facilitation by another entity in the sample extraction process, a sample kit can be provided to the subject. In the example, the sample kit can include one or more swabs for sample acquisition, one or more containers configured to receive the swab(s) and/or other biological sampling mediums for storage, instructions for sample provision and setup of a user account, elements configured to associate the sample(s) with the subject (e.g., barcode identifiers, tags, etc.), and a receptacle that allows the sample(s) from the subject to be delivered to a sample processing operation (e.g., by a mail delivery system). In another example, where the biological sample is extracted from the subject with the help of another entity, one or more samples can be collected in a clinical or research setting from the subject (e.g., during a clinical appointment). The biological sample can, however, be received from the subject in any other suitable manner.

Furthermore, in Block S150, processing and analyzing the biological sample from the subject is preferably performed in a manner similar to that of one of the embodiments, variations, and/or examples of sample reception described in relation to Block S110 above. As such, reception and processing of the biological sample in Block S150 can be performed for the subject using similar processes as those for receiving and processing biological samples used to generate the characterization process and/or the therapy model of the method 100, in order to provide consistency of process. However, biological sample reception and processing in Block S150 can alternatively be performed in any other suitable manner.

The method 100 can additionally or alternatively include Block S160, which recites: determining, with the characterization process, a characterization of the subject based upon processing a microbiome dataset (e.g., microbiome composition dataset, microbiome functional diversity dataset, etc.) derived from the biological sample of the subject. Block S160 functions to extract features from microbiome-derived data of the subject, and use the features as inputs into an embodiment, variation, or example of the characterization process (e.g., a characterization model) described in Block S130 above. Determining the characterization in Block S160 thus preferably includes identifying features and/or combinations of features associated with the microbiome composition and/or functional features of the subject, inputting the features into the characterization process, and receiving an output that characterizes the subject as belonging to one or more of: a behavioral group, a gender group, a dietary group, a disease-state group, and any other suitable group capable of being identified by the characterization process. Block S160 can further include generation of and/or output of a confidence metric associated with the characterization of the subject. For instance, a confidence metric can be derived from the number of features used to generate the characterization, relative weights or rankings of features used to generate the characterization, measures of bias in the characterization process, and/or any other suitable parameter associated with aspects of the characterization process.

In some variations of Block S160, features extracted from the microbiome dataset of the subject can be supplemented with survey-derived and/or medical history-derived features from the subject, which can be used to further refine the characterization process of Block S130. However, the microbiome dataset of the subject can additionally or alternatively be used in any other suitable manner to enhance the models of the method 100. In a variation, Block S160 can include generating values for features selected based on feature-selection rules (e.g., Clostridium-associated condition feature-selection rules), and using the values to characterize the subject. Such processes can confer improvements in the processing system by improving feature extraction processing speed by extracting only a subset of a set of features (e.g., microbiome composition features, microbiome functional diversity features, etc.) based on feature-selection rules (e.g., used in determining the subset of features used in training the corresponding characterization model), rather than generating each feature of the set of features. However, determining a characterization of the subject can be performed in any suitable manner.

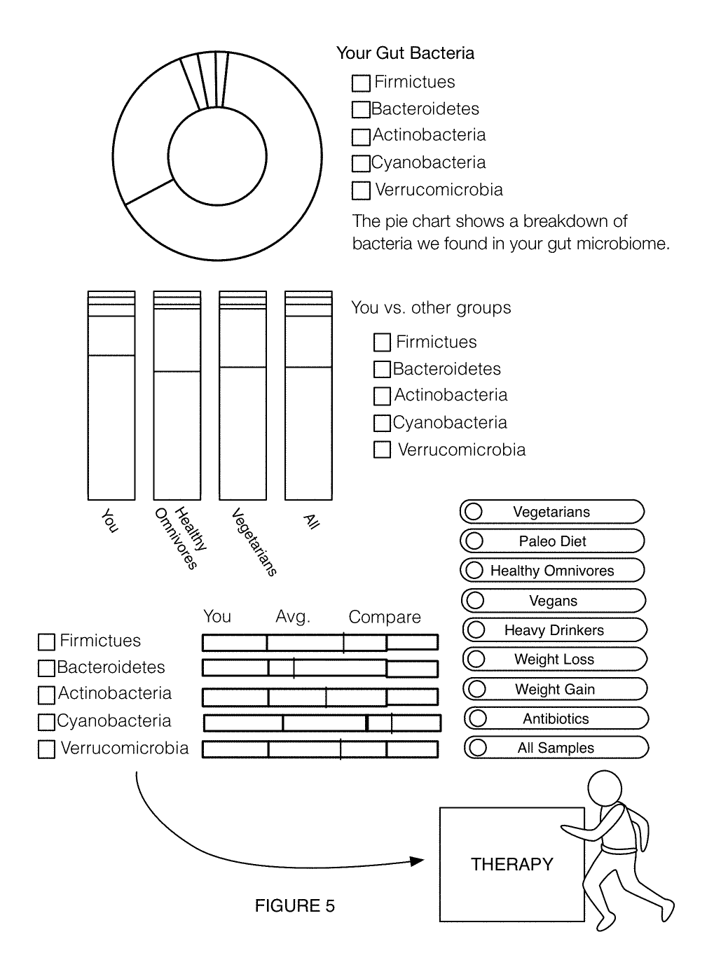

The method 100 can additionally or alternatively include Block S110, which recites: promoting a therapy (e.g., determined in Block S140) to the subject based upon the characterization and the therapy model, which functions to recommend or provide a personalized therapy to the subject, in order to shift the microbiome composition and/or functional features of the subject toward a desired equilibrium state. Block S110 can include provision of a customized therapy to the subject according to their microbiome composition and functional features, as shown in FIG. 5, where the customized therapy is a formulation of microorganisms configured to correct dysbiosis characteristic of subjects having the identified characterization. As such, outputs of Block S140 can be used to directly promote a customized therapy formulation and regimen (e.g., dosage, usage instructions) to the subject based upon a trained therapy model. Additionally or alternatively, therapy provision can include recommendation of available therapeutic measures configured to shift microbiome composition and/or functional features toward a desired state. In variations, available therapeutic measures can include one or more of: consumables (e.g., food items, beverage items, etc.), topical therapies (e.g., lotions, ointments, antiseptics, etc.), nutritional supplements (e.g., vitamins, minerals, fiber, fatty acids, amino acids, prebiotics, etc.), medications, antibiotics, bacteriophages, fecal matter transplant, and any other suitable therapeutic measure. For instance, a combination of commercially available probiotic supplements can include a suitable probiotic therapy for the subject according to an output of the therapy model.

Additionally or alternatively, in a specific example, the therapy of Block S110 can include a bacteriophage-based therapy. In more detail, one or more populations (e.g., in terms of colony forming units) of bacteriophages specific to a certain bacteria (or other microorganism) represented in the subject can be used to down-regulate or otherwise eliminate populations of the certain bacteria. As such, bacteriophage-based therapies can be used to reduce the size(s) of the undesired population(s) of bacteria represented in the subject. Complementarily, bacteriophage-based therapies can be used to increase the relative abundances of bacterial populations not targeted by the bacteriophage(s) used.

Therapy provision in Block S110 can include provision of notifications to a subject regarding the recommended therapy and/or other forms of therapy. Notifications can be provided to a subject by way of an electronic device (e.g., personal computer, mobile device, tablet, wearable, head-mounted wearable computing device, wrist-mounted wearable computing device, etc.) that executes an application, web interface, and/or messaging client configured for notification provision. In one example, a web interface of a personal computer or laptop associated with a subject can provide access, by the subject, to a user account of the subject, where the user account includes information regarding the user's characterization, detailed characterization of aspects of the user's microbiome, and notifications regarding suggested therapeutic measures generated in Blocks S140 and/or S110. In another example, an application executing at a personal electronic device (e.g., smart phone, smart watch, head-mounted smart device) can be configured to provide notifications (e.g., at a display, haptically, in an auditory manner, etc.) regarding therapy suggestions generated by the therapy model of Block S110. Notifications and/or probiotic therapies can additionally or alternatively be provided directly through an entity associated with a subject (e.g., a caretaker, a spouse, a significant other, a healthcare professional, etc.). In some further variations, notifications can additionally or alternatively be provided to an entity (e.g., healthcare professional) associated with a subject, where the entity is able to administer the therapy measure (e.g., by way of prescription, by way of conducting a therapeutic session, etc.). Notifications can, however, be provided for therapy administration to a subject in any other suitable manner.

Promoting a therapy in Block S110 can include controlling a therapy system (e.g., a communications system, an application executable on a user device, a medical device, a user device, etc.) to facilitate promotion of the therapy. Controlling a therapy system can include generating control instructions (e.g., at a processing system) for the therapy system, and operating the therapy system based on the control instructions (e.g., through transmitting the control instructions to the therapy system to execute). In an example, promoting a therapy can include controlling an administration system for consumables (e.g., an automated medication pillbox, a probiotic administration system) to distribute the consumable according to a regimen (e.g., by scheduling regimen reminders at the administration system; prompting the subject to take particular consumables; etc.) specified by a therapy. However, promoting a therapy can be performed in any suitable manner.

In some variations, the method 100 can additionally or alternatively include Block S180, which recites: monitoring effectiveness of the therapy for the subject, based upon processing biological samples, to assess microbiome composition and/or functional features for the subject at a set of time points associated with the probiotic therapy. Block S180 functions to gather additional data regarding positive effects, negative effects, and/or lack of effectiveness of a probiotic therapy suggested by the therapy model for subjects of a given characterization, where the additional data can be used, for example, to generate and/or update one or more characterization models, therapy models, and/or other suitable models. Monitoring of a subject during the course of a therapy promoted by the therapy model (e.g., by receiving and analyzing biological samples from the subject throughout therapy, by receiving survey-derived data from the subject throughout therapy) can thus be used to generate a therapy-effectiveness model for each characterization provided by the characterization process of Block S130, and each recommended therapy measure provided in Blocks S140 and S110.

In Block S180, the subject can be prompted to provide additional biological samples at one or more key time points of a therapy regimen that incorporates the therapy, and the additional biological sample(s) can be processed and analyzed (e.g., in a manner similar to that described in relation to Block S110) to generate metrics characterizing modulation of the subject's microbiome composition and/or functional features. For instance, metrics related to one or more of: a change in relative abundance of one or more taxonomic groups represented in the subject's microbiome at an earlier time point, a change in representation of a specific taxonomic group of the subject's microbiome, a ratio between abundance of a first taxonomic group of bacteria and abundance of a second taxonomic group of bacteria of the subject's microbiome, a change in relative abundance of one or more functional families in a subject's microbiome, and any other suitable metrics can be used to assess therapy effectiveness from changes in microbiome composition and/or functional features. Additionally or alternatively, survey-derived data from the subject, pertaining to experiences of the subject while on the therapy (e.g., experienced side effects, personal assessment of improvement, etc.) can be used to determine effectiveness of the therapy in Block S180. However, monitoring effectiveness of one or more therapies can be performed in any suitable manner.

The method 100 can, however, include any other suitable blocks or steps configured to facilitate reception of biological samples from subjects, processing of biological samples from subjects, analyzing data derived from biological samples, and generating models that can be used to provide customized diagnostics and/or probiotic-based therapeutics according to specific microbiome compositions and/or functional features of subjects.

The method 100 and/or system of the embodiments can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a patient computer or mobile device, or any suitable combination thereof. Other systems and methods of the embodiments can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor, though any suitable dedicated hardware device can (alternatively or additionally) execute the instructions.

The FIGURES illustrate the architecture, functionality and operation of possible implementations of systems, methods and computer program products according to preferred embodiments, example configurations, and variations thereof. In this regard, each block in the flowchart or block diagrams may represent a module, segment, step, or portion of code, which includes one or more executable instructions for implementing the specified logical function(s). It should also be noted that, in some alternative implementations, the functions noted in the block can occur out of the order noted in the FIGURES. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The embodiments include every combination and permutation of the various system components and the various method processes, including any variations, examples, and specific examples.

As a person skilled in the art will recognize from the previous detailed description and from the figures and claims, modifications and changes can be made to the embodiments of the invention without departing from the scope of this invention as defined in the following claims.

1. A system for characterizing a Clostridium-associated condition in relation to a user, the system comprising:
a handling network operable to receive containers comprising material from a set of users, the handling network comprising a sequencing system operable to determine microbiome sequences from sequencing the material;
a processing system operable to:
generate a microbiome composition dataset and a microbiome functional diversity dataset based on the microbiome sequences;
receive a supplementary dataset associated with the Clostridium-associated condition for the set of users;
transform the supplementary dataset and features extracted from the microbiome composition dataset and the microbiome functional diversity dataset into a characterization model for the Clostridium-associated condition; and
a therapy system operable to promote a therapy to the user based on characterizing the user in relation to the Clostridium-associated condition using the characterization model.

2. The system of claim 1, wherein the processing system is further operable to:
obtain a set of Clostridium-associated feature-selection rules correlating the Clostridium-associated condition to a subset of microbiome composition features and a subset of microbiome functional diversity features; and
generate the features based on evaluating the microbiome composition dataset and the microbiome functional diversity dataset against the set of Clostridium-associated feature-selection rules.

3. The system of claim 2, wherein the set of Clostridium-associated feature-selection rules improve the processing system by facilitating decreased processing time to transform the supplementary dataset and the features into the characterization model.

4. The system of claim 2, wherein the subset of microbiome functional diversity features comprises at least one of: a cluster of orthologous group of proteins feature, a genomic functional feature, a taxonomic feature, a chemical functional feature, and a systemic functional feature.

5. The system of claim 1,
wherein the Clostridium-associated condition comprises a Clostridium difficile Ribotype 027 strain infection comprising at least one of sepsis and colitis, and
wherein the associated features comprise at least one of the following: a microbiome functional diversity feature associated with bile acid metabolism, and a microbiome composition feature associated with a relative abundance of Bacteroidetes, Firmicutes, and Proteobacteria.

6. The system of claim 1, wherein the features comprise Kyoto Encyclopedia of Genes and Genomes (KEGG) functional features associated with at least one of: pentose phosphate pathway, gluconeogenesis, and carbon fixation.

7. The system of claim 1, further comprising an interface operable to improve display of Clostridium-associated condition information derived from the characterization model, wherein the Clostridium-associated condition information comprises a microbiome composition for the user relative to user group sharing a demographic characteristic, and wherein the microbiome composition comprises taxonomic groups comprising at least one of Clostridium difficile, Clostridium botulinum, and Clostridium perfringens.

8. The system of Claim 7, wherein the Clostridium-associated condition information comprises a risk of infection for the user relative to the user group, wherein the risk of infection is associated with at least one of: the taxonomic groups and functional features, and wherein the therapy is operable to reduce the risk of infection.

9. The system of claim 1, further comprising a sample kit comprising the containers, wherein the handling network is operable to deliver the containers to the set of users, and wherein the handling network further comprises a library preparation system operable to fragment and perform multiplex amplification on the material using primers compatible with microbiome targets associated with the Clostridium-associated condition.

10. A method for characterizing a Clostridium difficile (C. difficile) associated condition in relation to a user, the method comprising:
generating a microbiome composition dataset and a microbiome functional diversity dataset based on nucleic acid sequences derived from material samples from a set of users;
receiving a supplementary dataset informative of the C. difficile associated condition for the set of users;
obtaining a set of C. difficile associated feature-selection rules correlating the C. difficile associated condition to a subset of microbiome composition features and a subset of microbiome functional diversity features;
generating a feature set based on evaluating the microbiome composition dataset and the microbiome functional diversity dataset against the set of C. difficile associated feature-selection rules;
applying the feature set with the supplementary dataset to generate a characterization model for the C. difficile associated condition;
generating a characterization of the user in relation to the C. difficile associated condition using the characterization model; and
promoting a therapy to the user based on the characterization.

11. The method of claim 10, wherein the therapy is operable to facilitate modification of a user microbiome composition and a user microbiome functional diversity associated with the C. difficile associated condition, wherein promoting the therapy comprises controlling a therapy system to promote the therapy.

12. The method of claim 10, wherein generating the associated feature set comprises generating a set of microbiome feature vectors for the set of users based on the subset of microbiome composition features and the subset of microbiome functional diversity features, and wherein applying the feature set comprises training the characterization model with the set of microbiome feature vectors.

13. The method of claim 10, further comprising:
fragmenting and amplifying nucleic acid material derived from microorganisms in the sample material;
sequencing, with any suitable sequencing system, the nucleic acid material to determine the nucleic acid sequences; and
determining alignments between the nucleic acid sequences and reference sequences associated with the C. difficile associated condition, wherein generating the microbiome composition dataset and the microbiome functional diversity dataset is based on the alignments.

14. The method of claim 10, wherein the C. difficile associated condition is a C. difficile infection comprising at least one of sepsis and colitis, and wherein the characterization of the user comprises a diagnostic analysis for the C. difficile infection.

15. The method of claim 14, wherein the subset of microbiome functional diversity features comprises a functional feature associated with bile acid metabolism, and wherein generating the diagnostic analysis is based on using the characterization model with the associated functional feature.

16. The method of claim 14, wherein the supplementary dataset comprises biometric sensor data informative of the C. difficile infection, and wherein the set of C. difficile associated feature-selection rules correlates the C. difficile infection to a biometric feature derived from the biometric sensor data.

17. The method of claim 10, wherein the C. difficile associated condition comprises a C. difficile infection risk, and wherein comprises a therapy operable to facilitate modification of a user microbiome composition to reduce the C. difficile infection risk.

18. The method of claim 17, wherein the subset of microbiome composition features comprises a composition feature associated with a relative abundance of Bacteroidetes, Firmicutes, and Proteobacteria, wherein generating the characterization comprises determining the C. difficile infection risk based on using the characterization model with the composition feature, and wherein the therapy is operable to modify the relative abundance of Bacteroidetes, Firmicutes, and Proteobacteria to reduce the C. difficile infection risk.

19. The method of claim 17, wherein the supplementary dataset comprises antibiotic regimen data associated with the set of users, and wherein applying the associated feature set comprises applying the associated feature set with the antibiotic regimen data to generate the characterization model.

20. The method of claim 10, wherein the C. difficile associated condition comprises presence of C. difficile Ribotype 027 strain, and wherein generating the characterization comprises determining the presence of the C. difficile Ribotype 027 strain in a user microbiome composition.

21. The method of claim 19, wherein the associated feature set comprises a composition feature associated with a set of taxa comprising at least one of: Clostridium (genus), Clostridiaceae (family), and Firmicutes (phylum), and wherein determining the presence of the C. difficile Ribotype 027 strain comprises processing the characterization model with the composition associated feature.

22. The method of claim 20, wherein the associated feature set comprises a composition feature associated with a set of taxa comprising at least one of: Flavonifractor plautii (species), Bifidobacterium longum (species), Bacteroides fragilis (species), Bifidobacterium bifidum (species), Erysipelatoclostridium ramosum (species), Parabacteroides distasonis (species), Bacteroides vulgatus (species), Faecalibacterium prausnitzii (species), Blautia sp. YHC-4 (species), Blautia faecis (species), Bacteroides acidifaciens (species), Collinsella aerofaciens (species), Anaerostipes caccae (species), bacterium NLAE-zl-P855 (species), Bacteroides thetaiotaomicron (species), Bacteroides vulgatus (species), Bacteroides xylanisolvens (species), Bilophila wadsworthia (species), Blautia product (species), Clostridium clostridioforme (species), Clostridium hathewayi (species), Clostridium innocuum (species), Clostridium symbiosum (species), Eggerthella lenta (species), Escherichia coli (species), Haemophilus parainfluenzae (species), Intestinibacter bartlettii (species), Ruminococcus gnavus (species) and Ruminococcus torques (species); and wherein determining the presence of the C. difficile strain comprises processing the characterization model with the composition associated feature.

23. The method of claim 20, wherein the associated feature set comprises a composition feature associated with a set of taxa comprising at least one of: Roseburia (genus), Veillonella (genus), Kluyvera (genus), Sarcina (genus), Subdoligranulum (genus), Bifidobacterium (genus), Faecalibacterium (genus), Bilophila (genus), Lactobacillus (genus), Eubacterium (genus), Parabacteroides (genus), Akkermansia (genus), Dorea (genus), Bacteroides (genus), Moryella (genus), Anaerotruncus (genus), Enterococcus (genus), Eggerthella (genus), Collinsella (genus), Anaerobacter (genus), Megasphaera (genus), Alistipes (genus), Intestinimonas (genus), Streptococcus (genus), Flavonifractor (genus), Clostridium (genus), Peptoclostridium (genus), Pseudobutyrivibrio (genus), Erysipelatoclostridium (genus), Anaerostipes (genus), Blautia (genus), Escherichia-Shigella (genus), Haemophilus (genus), Hungatella (genus), Intestinibacter (genus) and Lachnoclostridium (genus); and wherein determining the presence of the C. difficile strain comprises processing the characterization model with the composition associated feature.

24. The method of claim 20, wherein the associated feature set comprises a composition feature associated with a set of taxa comprising at least one of: Ruminococcaceae (family), Enterobacteriaceae (family), Coriobacteriaceae (family), Lactobacillaceae (family), Lachnospiraceae (family), Bifidobacteriaceae (family), Eubacteriaceae (family), Verrucomicrobiaceae (family), Bacteroidaceae (family), Oscillospiraceae (family), Enterococcaceae (family), Rikenellaceae (family), Bradyrhizobiaceae (family), Clostridiaceae (family), Peptostreptococcaceae (family), Veillonellaceae (family), Christensenellaceae (family), Erysipelotrichaceae (family) and Streptococcaceae (family); and wherein determining the presence of the C. difficile strain comprises processing the characterization model with the composition associated feature.

25. The method of claim 20, wherein the associated feature set comprises a composition feature associated with a set of taxa comprising at least one of: Enterobacteriales (order), Clostridiales (order), Coriobacteriales (order), Bifidobacteriales (order), Verrucomicrobiales (order), Selenomonadales (order), Erysipelotrichales (order), Lactobacillales (order); and wherein determining the presence of the C. difficile strain comprises processing the characterization model with the composition associated feature.

26. The method of claim 10, wherein the associated feature set comprises a composition feature associated with a set of taxa comprising at least one of: Clostridia (class), Actinobacteria (class), Verrucomicrobiae (class), Alphaproteobacteria (class), Deltaproteobacteria (class), Negativicutes (class), Erysipelotrichia (class), Gammaproteobacteria (class), Bacilli (class); and wherein determining the presence of the C. difficile strain comprises processing the characterization model with the composition associated feature.

27. The method of claim 10, wherein the associated feature set comprises a composition feature associated with a set of taxa comprising at least one of: Proteobacteria (phylum), Actinobacteria (phylum), Verrucomicrobia (phylum) and Firmicutes (phylum); and wherein determining the presence of the C. difficile strain comprises processing the characterization model with the composition associated feature.

28. The method of claim 10, wherein the feature set comprises Kyoto Encyclopedia of Genes and Genomes (KEGG) functional features associated with at least one of: pentose phosphate pathway, gluconeogenesis, and carbon fixation, and wherein generating the characterization comprises processing the characterization model with the KEGG functional features.

29. The method of claim 10, wherein the feature set comprises Kyoto Encyclopedia of Genes and Genomes (KEGG) functional features associated with at least one of: Translation; Metabolism; Environmental Adaptation; Replication and Repair; Signaling Molecules and Interaction; Cellular Processes and Signaling; Energy Metabolism; Cell Growth and Death; Amino Acid Metabolism; Nucleotide Metabolism; Infectious Diseases; Nervous System; Signal Transduction; Endocrine System; Metabolism of Other Amino Acids; Carbohydrate Metabolism; Metabolism of Cofactors and Vitamins; Folding, sorting and Degradation; Membrane Transport; Metabolism of Terpenoids and Polyketides; Xenobiotics Biodegradation and Metabolism; Cell Motility; Metabolic Disease; Enzyme families and Biosynthesis of Other Secondary Metabolites; and wherein generating the characterization comprises processing the characterization model with the KEGG functional features.

30. The method of claim 10, wherein the feature set comprises Kyoto Encyclopedia of Genes and Genomes (KEGG) functional features associated with at least one of: Ribosome Biogenesis; Peptidoglycan biosynthesis; Chromosome; Inorganic ion transport and metabolism; Amino acid related enzymes; Amino acid metabolism; Ribosome; Aminoacyl-tRNA biosynthesis; Other ion-coupled transporters; Nitrogen metabolism; Photosynthesis; Translation factors; Photosynthesis proteins; Pantothenate and CoA biosynthesis, Plant-pathogen interaction, Homologous recombination, Terpenoid backbone biosynthesis, Phosphotransferase system (PTS); Bacterial toxins; Glyoxylate and dicarboxylate metabolism; DNA repair and recombination proteins; Translation proteins; Polycyclic aromatic hydrocarbon degradation; Biosynthesis and biodegradation of secondary metabolites; Tuberculosis; Pyrimidine metabolism; Cytoskeleton proteins; Protein export; Carbohydrate metabolism; One carbon pool by folate; RNA polymerase; Thiamine metabolism; Phenylalanine; tyrosine and tryptophan biosynthesis; Valine, leucine and isoleucine biosynthesis, Pentose and glucuronate interconversions; Cell cycle—Caulobacter; Butirosin and neomycin biosynthesis; DNA replication proteins; Base excision repair; Cell motility and secretion; Nucleotide excision repair; Nicotinate and nicotinamide metabolism; Glutathione metabolism; Zeatin biosynthesis; Vibrio cholerae pathogenic cycle; Alzheimer's disease; Mismatch repair; Protein folding and associated processing; Lysine biosynthesis; Fatty acid biosynthesis; Other transporters; Limonene and pinene degradation; Sulfur relay system; Glutamatergic synapse; Methane metabolism; Lipid biosynthesis proteins; Cs-Branched dibasic acid metabolism; Lysine degradation; Prenyltransferases; Ribosome biogenesis in eukaryotes; Lipopolysaccharide biosynthesis proteins; Chaperones and folding catalysts; Tryptophan metabolism; Vitamin metabolism; D-Glutamine and D-Glutamate metabolism; Bacterial chemotaxis; Transcription machinery; Two-component system; Sporulation; Restriction enzyme; Carbon fixation in photosynthetic organisms; Drug metabolism—other enzymes; Alanine, aspartate and glutamate metabolism; Pores ion channels; Histidine metabolism; Arginine and proline metabolism; Peptidases; Riboflavin metabolism; Starch and sucrose metabolism; Primary immunodeficiency; Oxidative phosphorylation; Lipid metabolism; Transcription factors; D-Alanine metabolism; Streptomycin biosynthesis; Taurine and hypotaurine metabolism; DNA replication; ABC transporters; Glycerophospholipid metabolism; Valine, leucine and isoleucine degradation; beta-Alanine metabolism; Carbon fixation pathways in prokaryotes; Polyketide sugar unit biosynthesis; Naphthalene degradation; Glycerolipid metabolism; General function prediction only; Protein kinases; Pentose phosphate pathway; Vitamin B6 metabolism; Glycosyltransferases; Phosphatidylinositol signaling system; Fructose and mannose metabolism; Membrane and intracellular structural molecules; Fatty acid metabolism and Type I diabetes mellitus; and wherein generating the characterization comprises processing the characterization model with the KEGG functional features.
